(19) United States
(12) Patent Application Publication
(10) Pub. No.: US 2007/0269436 A1
(43) Pub. Date: Nov. 22, 2007

(54) Method of Antibody Production

(75) Inventor: Jianhe Chen, Enfield (AU)

Correspondence Address: SEED INTELLECTUAL PROPERTY LAW GROUP PLLC, 701 FIFTH AVE, SUITE 5400, SEATTLE, WA, 98104, US

(73) Assignee: Apollo Life Science Limited, Beaconsfield (AU)

(21) Appl. No.: 10/537,850

(22) PCT Filed: Dec. 10, 2003
(86) PCT No.: PCT/AU03/01655

§ 371 Date: Apr. 18, 2007

Related U.S. Application Data
(60) Provisional application No. 60/432,395, filed on Dec. 10, 2002.

(51) Int. Cl. A61K 39/395; C07K 16/28; A61K 38/20; A61K 38/19

(52) U.S. Cl. 424/139.1; 435/326; 435/377; 436/501; 530/387.9; 424/85.1; 424/85.2

(57) ABSTRACT

The present invention relates to a method for the in vitro production of antibody in an antigen specific manner and, more particularly, to the in vitro production of human antibody in an antigen specific manner. The present invention further extends to the antibody produced therefrom and to the antibody producing cells produced in accordance with the methods disclosed herein. The antibodies and antibody producing cells of the present invention are useful, inter alia, in a range of therapeutic, prophylactic and diagnostic applications.

### FIELD OF THE INVENTION

The present invention relates to a method for the in vitro production of antibody in an antigen specific manner and, more particularly, to the in vitro production of human antibody in an antigen specific manner. The present invention further extends to the antibody produced therefrom and to the antibody producing cells produced in accordance with the methods disclosed herein. The antibodies and antibody producing cells of the present invention are useful, inter alia, in a range of therapeutic, prophylactic and diagnostic applications.

### BACKGROUND OF THE INVENTION

Bibliographic details referred to by author in this specification are collected at the end of the description.

The reference to any prior art in this specification is not, and should not be taken as, an acknowledgment or any form of suggestion that that prior art forms part of the common general knowledge in Australia.

One of the means by which mammals are cleared of foreign pathogens is via the immunologically specific humoral response, wherein antibody produced by B cells neutralises and clears the pathogen.

The in vivo mechanism by which a B cell secreting a specific antibody against a T cell dependent antigen is achieved involves the functional co-ordination of three major types of cells, these being professional antigen presenting cells, specific activated T helper cells (CD4/Th2) and committed B cells. Each of these cells' proliferation, activation and differentiation is mediated by, inter alia, cytokines, cell membrane interactions and other microenvironmental factors.

Antigen presenting cells include macrophages which are differentiated from monocytes, dendritic cells and some B cells. These cells are highly specialized in terms of their role in antigen uptake, internalization, and peptide re-expansion and presentation together with MHCII molecules on the cell surface. The cytokines, IL13, IL6 and IL10, produced by dendritic cells, also act to activate T cells to which antigen has been presented as well as ultimately inducing the proliferation of and antibody production by CD40-activated B cells (Thurner et al. 1999).

Subsequently to CD4 Th2 cell activation, their membrane CD40L and other membrane bound proteins (CD28, CTLA4 etc.) are upregulated. These T cells are then able to bind with B cells through the complementary receptor(s). Such direct interaction to activate B cells allows T cell signal triggering of non-switched B cells (De Boer, A. et al. 1998).

B cells stimulated with an antigen can be induced to proliferate after CD40 ligation by activated T helper (Th) cells (Rousset, F. et al. 1991, Lane, P. et al. 1992 and Noelle, R. J. et al., 1992). The B cells then undergo gene rearrangement to produce specific antibody against the antigen. CD19⁺ B cell have been identified as the major source of secreting hybridomas (Enno Schmidt, Ulrich Zimmerman 2001 and A De Boer et al. 1998).

Accessory cells involved in the induction of antibody production include fibroblasts, epithelial cells, endothelial cells and synovial cells. These cells are known to permit activated T cells to survive. Further, they provide specific and non-specific signals which augment B cell proliferation and antibody production (Akbar et al. 1993).

The seminal work of George Köhler and Cesar Milstein (1975, Nature 256: 495-497) described the first method of producing monoclonal antibodies, in vitro, which recognise a single determinant or epitope of an antigen. This work heralded a significant technical breakthrough with important consequences both scientifically and commercially.

Traditionally, monoclonal antibodies are made by establishing an immortal mammalian cell line which is derived from a single immunoglobulin producing cell secreting one form of an antibody molecule with a particular specificity. Because the antibody-secreting mammalian cell line is immortal, the characteristics of the antibody are reproducible from batch to batch. The key properties of monoclonal antibodies are their specificity for a particular epitope of an antigen and the reproducibility with which they can be manufactured.

To date, most work has centred on the use of non-human, especially murine, monoclonal antibodies. While murine monoclonal antibodies provide valuable tools for the study of biological processes either in vitro or via murine models, there do exist limitations to their application. One of these impediments relates to the development of in vivo therapeutic and diagnostic applications for monoclonal antibodies in humans. Specifically, non-human immunoglobulins are inherently immunogenic when administered to humans. For example, when immunocompetent human patients are administered therapeutic doses of rodent monoclonal antibodies, the patients produce antibodies against the rodent immunoglobulin sequences. These human anti-mouse antibodies (HAMA) neutralise the therapeutic antibodies and can cause acute toxicity (the HAMA response). Hence, it is desirable to produce human immunoglobulins, which are reactive with specific antigens, for promising therapeutic and/or diagnostic targets.

Human antigen-specific antibodies are made by human lymphocytes. It is difficult to obtain immune human lymphocytes for immortalisation. One way to obtain human lymphocytes that produce specific antibodies is to immunise patients with the molecules against which it is desired to produce the antibodies. But such a procedure is ethically unacceptable. Another way to obtain immune human lymphocytes is to seek out patients suffering from specific tumors and infections, thereby gaining access to in vivo sensitized lymphocytes. However, this method is practically not feasible. Accordingly, no human in vivo system has been developed for the continuous and large scale production of human monoclonal antibodies.

Due to the potentially high commercial value, attempts have been made to devise in vitro systems and processes in order to produce human monoclonal antibodies in high quantities, but none have yet led to human monoclonal antibody production sufficient to fulfil the outstanding need. Accordingly, demand for the development of such processes and systems remains high.

This alternative to in vivo approaches is based on the in vitro immunisation of non-immune human lymphocytes. In such a system, many types of antigens can be employed. The in vitro immunised human lymphocytes can then be fused with myeloma or lymphoblastoid cells for immortalisation and thus continuous production of monoclonal antibodies. Immortalisation can also be achieved by transfection with a suitable viral or bacterial vector. Typically, however, yields and affinities of specific antibodies produced by these human systems have so far been too low for commercial use. Another problem encountered with these systems is the cytotoxic effect of cell subpopulations that are capable of suppressing the antigen-specific immunological response in vitro. Borrebaeck et al. (U.S. Pat. No. 5,567,610) have attempted to solve this problem by using lysosomotropic agents to kill all lysosome-containing cells (monocytes, macrophages, NK cells, etc.) in the cell population prior to in vitro immunisation.

While a limited number of processes for in vitro immunisation and the production of human monoclonal antibodies are presently available, there remains a need for more effective processes for this purpose.

In work leading up to the present invention it has been determined that current in vitro “immunisation” techniques fail to generate optimal B cell activation and antibody production levels due to what have now been surprisingly identified as previously unrecognised non-optimal features of the culturing methodology. For example, it has been determined that accepted dogma which recommends the complete eradication of lysosomal containing cells from the lymphocyte culture (in order to prevent suppression of the antigen specific response) in fact adversely impact on B cell activation due to the elimination of cellular subpopulations previously unrecognised as contributing to in vitro B cell activation. Further, the stimulation of dendritic cell differentiation during their co-culture with lymphoid cells has now been found to lead to inhibition of T cell proliferation and the induction of ongoing, unwanted apoptosis due to the adverse pleiotropic effects of the cytokines required to support dendritic cell differentiation.

Accordingly, there has now been developed a method of inducing antigen specific B cell activation which overcomes these and other problems, thereby leading to the development of a process which achieves a level of effectiveness previously unobtainable. The development of this method of inducing, in vitro, B cell activation and antibody production in an antigen specific manner therefore now facilitates the in vitro generation of antibody to any given antigen of interest in a species specific manner at a previously unobtainable qualitative and quantitative level. There is therefore facilitated the generation of specific antibody for a wide range of therapeutic, prophylactic and/or diagnostic applications. Still further, immortalisation of the antibody producing plasma cell of interest provides a means for the indefinite production of the antibody of interest and therefore the prospect of generating commercially viable quantities of the subject antibody.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The articles “a” and “an” are used herein to refer to one or to more than one (ie. to at least one) of the grammatical object of the article. By way of example, “an element” means one element or more than one element.

The subject specification contains amino acid sequence information prepared using the programme PatentIn Version 3.1, presented herein after the bibliography. Each amino acid sequence is identified in the sequence listing by the numeric indicator <210>followed by the sequence identifier (e.g. <210>1, <210>2, etc). The length, type of sequence (Protein etc) and source organism for each amino acid sequence is indicated by information provided in the numeric indicator fields <211>, <212> and <213>, respectively. Amino acid sequences referred to in the specification are identified by the indicator SEQ ID NO: followed by the sequence identifier (eg. SEQ ID NO:1, SEQ ID NO: 2, etc.). The sequence identifier referred to in the specification correlates to the information provided in numeric indicator <400>in the sequence listing, which is followed by the sequence identifier (eg. <400>1, <400>2, etc). That is SEQ ID NO:1 as detailed in the specification correlates to the sequence as <400>1 in the sequence listing.

One aspect of the present invention is directed to a method for the in vitro antigen specific activation of antibody producing cells, said method comprising the steps of:
(i) culturing a population of isolated, non-adherent mononuclear immune cells, which population comprises T helper cells or functional equivalent thereof, said antibody producing cells and a functionally insignificant number of lysosome-containing cells, for a time and under conditions sufficient to induce differentiation of said antibody producing cell;
(ii) culturing a population of adherent mononuclear immune cells for a time and under conditions sufficient to facilitate antigen presenting cell differentiation;
(iii) sequentially pulsing the cell population of step (i) with:
   an effective number of cells derived from cell population of step (ii), wherein the presentation of said antigen by said antigen presenting cells is facilitated; and
      a functionally significant number of lysosome-containing cells
for a time and under conditions sufficient to facilitate antigen specific activation of said antibody producing cells.

Another aspect of the present invention provides a method for the in vitro antigen specific activation of a B cell, said method comprising the steps of:
(i) culturing a population of isolated, non-adherent mononuclear immune cells, which population comprises T helper cells or functional equivalent thereof, said B cells and a functionally insignificant number of lysosome-containing cells, for a time and under conditions sufficient to induce differentiation of said B cell;
(ii) culturing a population of adherent mononuclear immune cells for a time and under conditions sufficient to facilitate antigen presenting cell differentiation;
(iii) sequentially pulsing the cell population of step (i) with:
   an effective number of cells derived from cell population of step (ii), wherein the presentation of said antigen by said antigen presenting cells is facilitated; and
      a functionally significant number of lysosome-containing cells
for a time and under conditions sufficient to facilitate antigen specific activation of said B cells.

In yet another aspect of the invention there is provided a method for the in vitro antigen specific activation of B cells, said method comprising the steps of:
(i) culturing a population of isolated, non-adherent mononuclear immune cells, which population comprises T helper cells or functional equivalent thereof, said B cell and a functionally insignificant number of lysosome-containing cells, for a time and under conditions sufficient to induce differentiation of said B cell;
(ii) culturing a population of adherent mononuclear immune cells for a time and under conditions sufficient to facilitate antigen presenting cell differentiation;
(iii) sequentially pulsing the cell population of step (i) with:
   an effective number of cells derived from cell population of step (ii), wherein the presentation of said antigen by said antigen presenting cells is facilitated; and
      a functionally significant number of lysosome-containing cells
for a time and under conditions sufficient to facilitate antigen specific activation of said B cells;
   wherein said B cells undergo differentiation to pre-plasma cells.

In still another aspect there is provided a method for the in vitro antigen specific activation of human B cells, said method comprising the steps of:
(i) culturing a population of isolated, non-adherent mononuclear human immune cells, which population comprises T helper cells or functional equivalent thereof, said B cells and a functionally insignificant number of lysosome-containing cells, for a time and under conditions sufficient to induce differentiation of said B cell;
(ii) culturing a population of adherent mononuclear human immune cells for a time and under conditions sufficient to facilitate antigen presenting cell differentiation;
(iii) sequentially pulsing the cell population of step (i) with:
   an effective number of cells derived from cell population of step (ii), wherein the presentation of said antigen by said antigen presenting cells is facilitated; and
      a functionally significant number of lysosome-containing cells
for a time and under conditions sufficient to facilitate antigen specific activation of said B cells;
   wherein said B cells undergo differentiation to pre-plasma cells.

In yet still another aspect there is provided a method for the in vitro antigen specific activation of human B cells, said method comprising the steps of:
(i) culturing a population of isolated, non-adherent human peripheral blood mononuclear cells, which population comprises T helper cells or functional equivalent thereof, said B cells and a functionally insignificant number of lysosome-containing cells, for a time and under conditions sufficient to induce differentiation of said B cell;
(ii) culturing a population of adherent human peripheral blood mononuclear cells for a time and conditions to facilitate antigen presenting cell differentiation;
(iii) sequentially pulsing the cell population of step (i) with:
   an effective number of cells derived from cell population of step (ii), wherein the presentation of said antigen by said antigen presenting cells is facilitated; and
   a functionally significant number of lysosome-containing cells
for a time and under conditions sufficient to facilitate antigen specific activation of said B cells;
   wherein said B cells undergo differentiation to pre-plasma cells.

In a further aspect there is provided a method for the in vitro antigen specific activation of human B cells said method comprising the steps of:
(i) culturing a population of isolated, non-adherent human mononuclear immune cells, which population comprises Th2 cells or functional equivalent thereof, said B cells and a functionally insignificant number of lysosome-containing cells, for a time and under conditions sufficient to induce differentiation of said B cells;
(ii) culturing a population of adherent human mononuclear immune cells for a time and under conditions sufficient to facilitate antigen presenting cell differentiation;
(iii) sequentially pulsing the cell population of step (i) with:
   an effective number of cells derived from cell population of step (ii), wherein the presentation of said antigen by said antigen presenting cells is facilitated; and
      a functionally significant number of lysosome-containing cells
for a time and under conditions sufficient to facilitate antigen specific activation of said B cells;
   wherein said B cells undergo differentiation to pre-plasma cells.

Another further aspect of the present invention provides a method for the in vitro antigen specific activation of human B cells said method comprising the steps of:
(i) culturing a population of isolated, non-adherent mononuclear immune cells, which population comprises Th2 cells or functional equivalent thereof, said B cells and a functionally insignificant number of lysosome-containing cells, for a time and under conditions sufficient to induce differentiation of said B cells;
(ii) culturing a population of adherent human mononuclear immune cells, for a time and under conditions sufficient to facilitate dendritic cell differentiation;
(iii) sequentially pulsing the cell population of step (i) with:
   an effective number of cells derived from cell population of step (ii), wherein the presentation of said antigen by said dendritic cells is facilitated; and
      a functionally significant number of lysosome-containing cells
for a time and under conditions sufficient to facilitate antigen specific activation of said B cells;
   wherein said B cells undergo differentiation to pre-plasma cells.

Yet another further aspect of the present invention is directed to a method for the in vitro antigen specific activation of human B cells, said method comprising the steps of:
(i) co-culturing a population of isolated, non-adherent human mononuclear immune cells, which population comprises Th2 cells or functional equivalent thereof, said B cells and a functionally insignificant number of lysosome-containing cells, together with an effective amount of one or more growth factors and anti-CD40 ligand, for a time and under conditions sufficient to induce differentiation of said B cells;
(ii) co-culturing a population of adherent human mononuclear immune cells together with an effective amount of one or more growth factors, for a time and under conditions sufficient to facilitate dendritic cell differentiation;
(iii) sequentially pulsing the cell population of step (i) with:
   an effective number of cells derived from the cell population of step (ii), wherein the presentation of said antigen by said dendritic cells is facilitated; and
   substantially 10% lysosome-containing cells;
for a time and under conditions sufficient to facilitate antigen specific activation of said B cells;
   wherein said B cells undergo differentiation to a pre-plasma cells.

Still another further aspect of the present invention is directed to a method for the in vitro antigen specific activation of human B cells, said method comprising the steps of:
(i) co-culturing a population of isolated, non-adherent human mononuclear immune cells, which population comprises Th2 cells or functional equivalent thereof, said B cells and a functionally insignificant number of lysosome-containing cells, together with an effective amount of IL4, GM-CSF and anti-CD40 ligand, for a time and under conditions sufficient to induce differentiation of said B cells;
(ii) co-culturing a population of adherent human mononuclear immune cells together with IL-4, GM/CSF and/or TNFα, for a time and under conditions sufficient to facilitate dendritic cell differentiation;
(iii) sequentially pulsing the cell population of step (i) with:
   an effective number of cells derived from the cell population of step (ii), wherein the presentation of said antigen by said dendritic cells is facilitated; and
   substantially 10% lysosome-containing cells;
for a time and under conditions sufficient to facilitate antigen specific activation of said B cells;
   wherein said B cells undergo differentiation to a pre-plasma cells.

In still yet another further aspect there is provided a method for the in vitro activation of Hepatitis B specific human B cells, said method comprising the steps of:
(i) co-culturing a population of isolated, non-adherent human mononuclear immune cells, which population comprises Th2 cells or functional equivalent thereof, said B cells and a functionally insignificant number of lysosome-containing cells, together with an effective amount of IL-4, GM-CSF and anti-CD40 ligand, for a time and under conditions sufficient to induce differentiation of said B cells;
(ii) co-culturing. a population of adherent human mononuclear immune cells together with IL-4, GM-CSF and/or TNFα, for a time and under conditions sufficient to facilitate dendritic cell differentiation;
(iii) sequentially pulsing the cell population of step (i) with:
   an effective number of cells derived from the cell population of step (ii), wherein the presentation of Pre-S2 HBV by said dendritic cells is facilitated; and
   substantially 10% lysosome-containing cells;
for a time and under conditions sufficient to facilitate antigen specific activation of said B cells;
   wherein said B cells undergo differentiation to pre-plasma cells.

Another aspect of the present invention should be understood to extend to a method of therapeutically and/or prophylactically treating a subject, said method comprising administering to said subject an effective amount of antibody or derivative, homologue, analogue, chemical equivalent or mimetic of said antibody wherein said antibody is produced by the method of the present invention.

The present invention also extends to the use of antibody or derivative, homologue, analogue, equivalent or mimetic produced in accordance with the method of the present invention in relation to diagnostic applications.

Still another aspect of the present invention is directed to the antibody or derivative, homologue, analogue, equivalent or mimetic and activated antibody producing cells produced in accordance with the methods defined hereinbefore.

Single and three letter abbreviations used throughout the specification are defined in Table 1.

TABLE 1
Single and three letter amino acid abbreviations

| | Three-letter | |
|---|---|---|
| Amino Acid | Abbreviation | One-letter Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| As defined | Xaa | X |

### BRIEF DESCRIPTION OF THE DRAWINGS

FIG. 1 provides a detailed schematic representation of the process exemplified herein for the in vitro production of human antibodies in an antigen specific manner. Mononuclear cells were obtained from spleen or peripheral blood immediately when a tissue arrived. 4×10⁷ cells were set aside for producing endogenous cytokines called culture 3 here. 10 ug/ml PHA-Lock or 20 ng/ml PMA & 1 ug/ml ionomycin were washed off after 4 hours or overnight stimulation. Culture I was added with 30 ng/ml IL4 and 30-ng/ml GM-CSF at day 0, 4 and 8 and 100 unit/ml TNFα at day 7. Culture 2 was maintained in 5 ng/ml IL-4, 5 ng/ml GM-CSF and 0.5 ug/ml anti-CD40 in growth medium until day 4. At day 4 one third of the cells from culture 1 were mixed with culture 2 in 10% supernatant of culture 3 and 90% fresh culture medium. At day 7 another one third of the cells from culture 1 were placed in fresh culture medium with the addition of 5 ng/ml IL-4, 5 ng/ml GM-CSF and 0.5 ug/ml anti-CD40. At day 11 the final one third of the cells from culture 1 were placed in fresh culture medium with the addition of 5 ng/ml IL-4, 5 ng/ml GM-CSF, but without 0.5 ug/ml anti-CD40. All cells from culture 1 were pulsed with antigen for 2 hours before being added to cells from culture 2 (FIG. 3). Confirmation of the generation of specific antibody producers was done by ELISPOT.

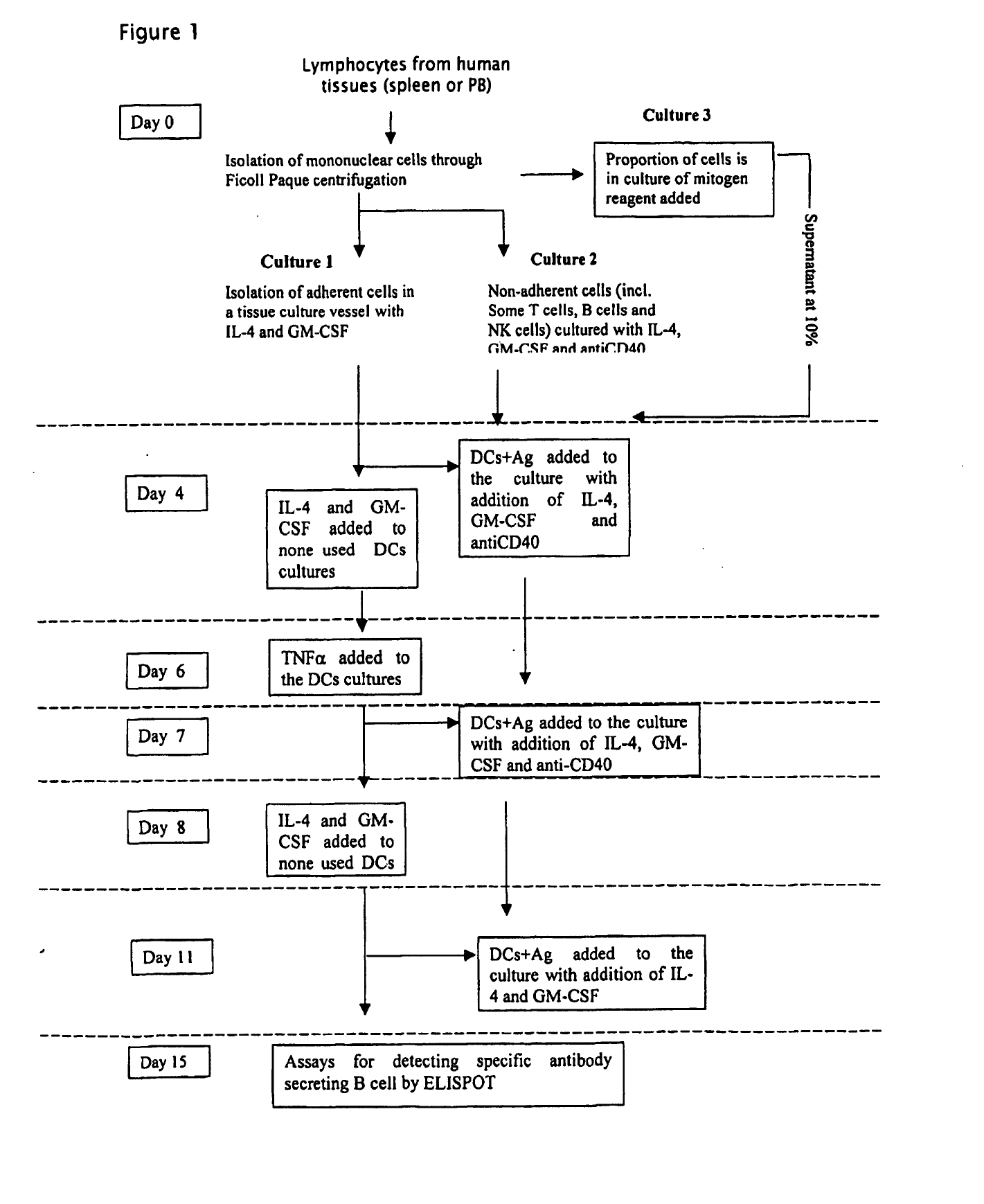

FIG. 2 provides an image of immunised cells, which were obtained from human spleen tissue, and subjected to the in vitro immunisation procedure described this patent. 4×10⁴ cells were seeded to D1, E1 and F1. 25 spots were counted from well D1 (which was PreS2 coated) as well as well El (which was PreS2-KLH coated). A mouse hybridoma cell line named H8, which secretes a well-characterised mouse anti-PreS2 antibody, was used as a positive control for the assay.

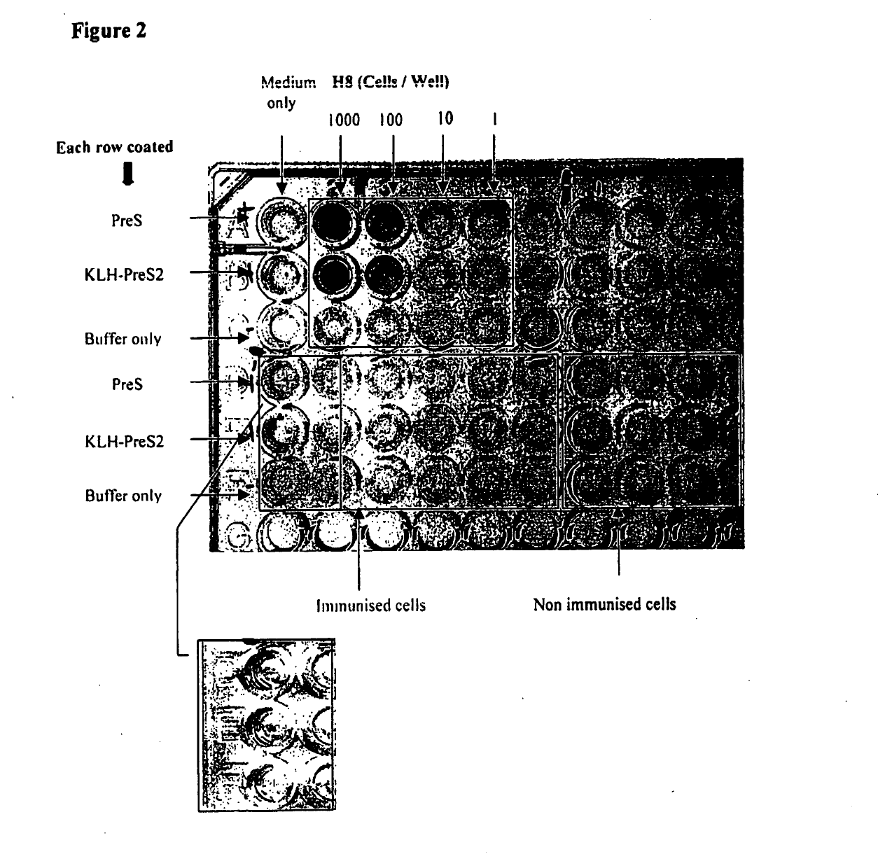

FIG. 3 provides an image of two examples of antigen uptake. Picture A: Cells were generated in adherent cell culture by stimulation with 11-4, GM-CSF and TNFα (viewed at 40× with light microscopy). Picture B: Cells were pulsed with PreS2-FITC at 5 ug/ml for 2 hours (viewed at 40× with fluorescence microscopy). Picture C: overlay of A and B.

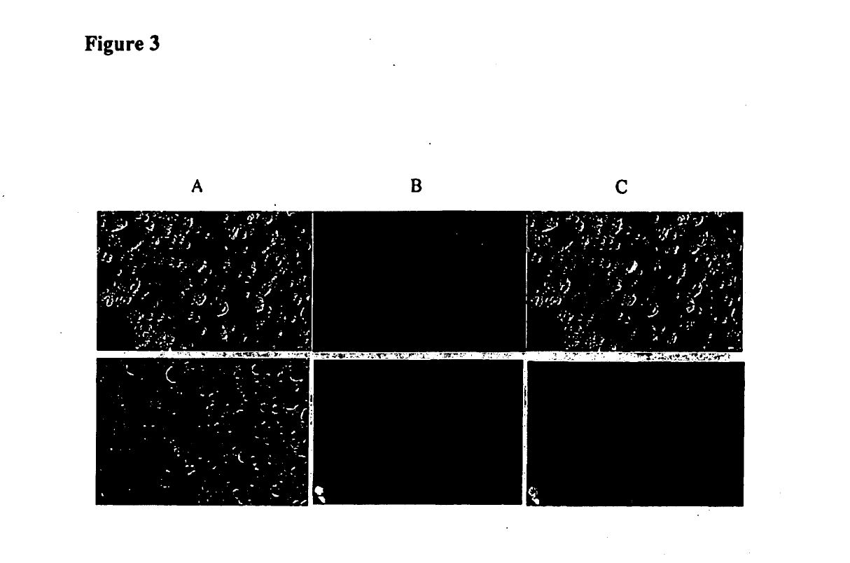

### DETAILED DESCRIPTION OF THE INVENTION

The present invention is predicated, in part, on the determination that currently existing methods directed to the in vitro activation of B cells in an antigen specific manner are far from optimal due to previously unrecognised problems inherent in the accepted culture methodology. In particular, it has now been determined that contrary to recommended practice:
(i) a percentage of lysosomal-containing cells are required to be co-cultured with the lymphoid population in order to facilitate T cell activation and B cell activation; and
(ii) dendritic cell differentiation must not be induced in co-culture with the lymphoid population since this both adversely impacts on T cell proliferation and differentiation and leads to the induction of lymphoid apoptosis.

Accordingly, one aspect of the present invention is directed to a method for the in vitro antigen specific activation of antibody producing cells, said method comprising the steps of:
(i) culturing a population of isolated, non-adherent mononuclear immune cells, which population comprises T helper cells or functional equivalent thereof, said antibody producing cells and a functionally insignificant number of lysosome-containing cells, for a time and under conditions sufficient to induce differentiation of said antibody producing cell;
(ii) culturing a population of adherent mononuclear immune cells for a time and under conditions sufficient to facilitate antigen presenting cell differentiation;
(iii) sequentially pulsing the cell population of step (i) with:
   an effective number of cells derived from cell population of step (ii), wherein the presentation of said antigen by said antigen presenting cells is facilitated; and
      a functionally significant number of lysosome-containing cells
for a time and under conditions sufficient to facilitate antigen specific activation of said antibody producing cells.

Reference to “antibody producing cells” should be understood as a reference to any cell which can, under appropriate stimulatory conditions, secrete antibody or derivative, mutant or variant thereof or a mutant or variant of such cells. “Variants” include, but are not limited to, cells exhibiting some but not all of the phenotypic features or functional activities of typical antibody producing cells. For example, a B cell variant includes B cells which either congenitally or due to intervention (such as recombinant, biochemical or as a result of the onset of a disease condition) exhibit some but not all of the phenotypic features or functional activities of a B cell. Alternatively, the subject cell may exhibit phenotypic features or functional activities in addition to those normally expressed by a B cell (for example, the B cell may have been fused with a cell type which introduces the functional attribute of immortality). “Mutants” include, but are not limited to, antibody producing cells which are genetically altered, for example transgenic cells. Such genetic alteration may occur spontaneously or it may be the result of recombinant or other intervention. The genetic modification may be for any purpose including, but not limited to, modifying the subject cell's immunoglobulin DNA or introducing or deleting all or part of a gene (whether that gene be an immunoglobulin gene or non-immunoglobulin gene—such as a growth factor gene). The person of skill in the art would understand that there is a degree of overlap between cells which could be described as “variants” and those which could be described as “mutants”.

It should also be understood that the subject antibody producing cells may be at any differentiation stage. Accordingly, the cells may be immature and therefore functionally incompetent in the absence of further differentiation. In this regard, highly immature cells such as stem-cells or colony forming unit cells, which retain the capacity to differentiate into a range of immune or non-immune cell types, should nevertheless be understood to satisfy the definition of “antibody producing cell” as utilised herein due to their capacity to differentiate into antibody producing cells under appropriate conditions. The method of the present invention incorporates a culturing step which facilitates the ongoing differentiation of antibody producing cells and, as a matter of routine procedure and to the extent that it may not inherently occur, could be adapted to incorporate a culturing step which facilitates the differentiation of highly immature antibody producing cells. Preferably, said antibody producing cell is a B cell.

The present invention therefore more particularly provides a method for the in vitro antigen specific activation of a B cell, said method comprising the steps of:
(i) culturing a population of isolated, non-adherent mononuclear immune cells, which population comprises T helper cells or functional equivalent thereof, said B cells and a functionally insignificant number of lysosome-containing cells, for a time and under conditions sufficient to induce differentiation of said B cell;
(ii) culturing a population of adherent mononuclear immune cells for a time and under conditions sufficient to facilitate antigen presenting cell differentiation;
(iii) sequentially pulsing the cell population of step (i) with:
   an effective number of cells derived from cell population of step (ii), wherein the presentation of said antigen by said antigen presenting cells is facilitated; and
      a functionally significant number of lysosome-containing cells
for a time and under conditions sufficient to facilitate antigen specific activation of said B cells.

Reference to “antibody or derivative, mutant or variant thereof” should be understood as a reference to all forms of any member of the immunoglobulin family of molecules and to derivatives thereof. Without limiting the present invention to any one theory or mode of action, the immunoglobulin family of molecules are currently believed to encompass five classes (isotypes) of immunoglobulin, being IgM, IgG, IgA, IgD and IgE. The immunoglobulins classed as IgG and IgA are further divided into the subclasses IgG1, IgG2, IgG3, IgG4, IgA1 and IgA2. Each individual antibody molecule exhibits unique complimentary determining region (CDR) domains on the variable regions of the immunoglobulin heavy and light chains, thereby attributing that immunoglobulin molecule with a highly specific epitope reactivity. It is currently believed that the human immune system generates in the excess of 10⁸ unique immunoglobulin epitope specificities. Reference to “antibody or derivative, mutant or variant thereof” includes, for example, all protein forms of these molecules or their derivatives, mutants or variants including, for example, any isoforms which may arise from alternative splicing of the DNA (during immunoglobulin rearrangement) or of the encoding mRNA. Also included in this definition are mutants, variants (such as polymorphic variants) or derivatives of these molecules. “Derivatives” include, for example, portions of the immunoglobulin molecule such as the Fab fragment, F(ab′)₂ fragment, Fc fragment or the individual heavy and light chains or fragments thereof (such as the variable or constant region domains) of which a B cell may have been genetically or otherwise modified to produce. Also included are immunoglobulin molecules which have spontaneously or otherwise (such as resulting from genetic manipulation) undergone a mutation at either the nucleic acid or amino acid sequence level. Still further, derivatives of immunoglobulin molecules, such as the functionally unique catalytic antibodies, which may be produced by a B cell either naturally or as a result of the genetic or other manipulation of the B cell are included within the scope of the present invention. Also included is the notion of species homologues which a given B cell may be engineered to produce. “Mutants” should be understood as a reference to antibody molecules which comprise, at either the amino acid encoding DNA level, amino acid or nucleic acid mutations, such as insertions, deletions, substitutions or other form of modification.

As detailed hereinbefore, the method of the present invention is directed to the in vitro “antigen specific” activation of antibody producing cells. By “antigen specific” is meant that the antibody producing cells comprising the heterogeneous population of non-adherent mononuclear immune cells are activated in an antigen specific manner. That is, predominantly only those antibody producing cells which will produce an antibody expressing a specificity directed to an epitope present on a given antigen will be activated, as opposed to the non-specific activation of the B cell population as a whole—as might occur in the presence of a mitogen or super antigen. Accordingly, although some antigens may express only one epitope, it is conceivable that an antigen of interest may express more than one epitope. It should therefore be understood that in such a situation it is likely that more than one specificity of antibody producing cell may be activated, which specificities are directed to the various epitopes comprising the antigen of interest. It should also be understood that to the extent that antibody cross reactivity may occur in respect of a given epitope, it is also conceivable that more than one specificity of antibody producing cell may be activated in response to a single epitope of interest.

In the context of the present invention, reference to the “activation” of an antibody producing cell should be understood as a reference to the induction of and including the differentiation and proliferation mechanisms which memory or virgin antibody producing cells are required to undergo in order to synthesise and/or secrete antibodies. In this regard, it should be understood that the method of the present invention may be utilised to produce an antibody producing cell of any differentiative stage of development subsequently to the time of its initial activation and through to terminal differentiation to a plasma cell and antibody secretion. In this regard, for example, virgin B cells are currently understood to produce IgM in response to an initial activation signal, which IgM production may switch to IgG isotype production pursuant to ongoing antigen stimulation. Accordingly, it may be desirable to utilise the method of the present invention such that IgM producing B cells are produced rather than driving post activation differentiation to the point that immunoglobulin class switching occurs. In another example, the method of the invention can be utilised to generate terminally differentiated B cells, that is plasma cells. In another preferred embodiment, it may be desirable to apply the method of the present invention in order to facilitate the isolation of activated B cells, in an antigen specific manner, which have not yet undergone terminal differentiation to plasma cells. For example, it may be desirable to isolate pre-plasma cells. Such cells are thought to be particularly useful for producing hybridomas. In some situations they are, in fact, thought to be better for producing hybridomas than plasma cells. These cells can be isolated by any suitable technique, which would be well known to those of skill in the art, such as FACS sorting based on cell surface molecule expression. In this regard, patterns of cell surface molecule expression are well known in the context of the B cell differentiation pathway and the person of skill in the art could design, as a matter of routine procedure, means of isolating specific cell populations based on expression of unique cell surface molecule expression. In another example, it may be desirable to isolate the memory B cells which are directly generated from naive B cells in accordance with the method of the present invention. Preferably, the method of the invention is directed to isolating CD19⁺/CD38⁺ pre-plasma cells. Accordingly, reference to “activation” herein should be understood to include the initial activation of the antibody producing cell (as hereinbefore defined) and all or part of the subsequent differentiative phase which the subject cell undergoes through to its terminal differentiation.

According to this preferred embodiment, there is provided the method for the in vitro antigen specific activation of B cells, said method comprising the steps of:
(i) culturing a population of isolated, non-adherent mononuclear immune cells, which population comprises T helper cells or functional equivalent thereof, said B cell and a functionally insignificant number of lysosome-containing cells, for a time and under conditions sufficient to induce differentiation of said B cell;
(ii) culturing a population of adherent mononuclear immune cells for a time and under conditions sufficient to facilitate antigen presenting cell differentiation;
(iii) sequentially pulsing the cell population of step (i) with:
   an effective number of cells derived from cell population of step (ii), wherein the presentation of said antigen by said antigen presenting cells is facilitated; and
      a functionally significant number of lysosome-containing cells
for a time and under conditions sufficient to facilitate antigen specific activation of said B cells;
   wherein said B cells undergo differentiation to pre-plasma cells.

Preferably, said pre-plasma cells are CD19⁺/CD38⁺.

As detailed hereinbefore, the present invention is predicated, in part, on the determination that the induction of in vitro B cell activation is best achieved where the generation of a dendritic cell population suitable for antigen presentation to T helper cells is not performed in co-culture with the subject T helper cells (in particular) and B cells as has been the practice prior to the advent of the present invention. Without limiting the present invention to any one theory or mode of action, it has been determined that the soluble factors required to induce and maintain dendritic cell differentiation and viability, respectively, in fact adversely impact on the differentiation and viability of certain subpopulations of lymphoid cells. For example, relatively high concentration of GM-CSF are useful for inducing dendritic cell differentiation. However, although GM-CSF can nevertheless also maintain B cell differentiation, at the high concentrations required to support dendritic cell cultures this cytokine has now been found to inhibit T helper cell differentiation and proliferation at a level which adversely impacts on the in vitro activation of B cells. In another example, whereas TNFα can induce the differentiation of immature dendritic cells to mature dendritic cells, in terms of non-dendritic cells the presence of this cytokine has now been determined to lead to ongoing and unwanted apoptosis at a level which also adversely impacts on the in vitro activation of B cells. Accordingly, the method of the present invention defines a parallel culturing system wherein the preparation of dendritic cells for co-culture with the lymphoid cultures of interest is conducted apart from said lymphoid culture. Dendritic cells are thereby introduced to the lymphoid culture upon reaching a particular differentiative stage and only at the most suitable time point.

The method of the present invention is exemplified herein with respect to the in vitro antigen specific activation of human spleen and peripheral blood derived B cells. However, it should be understood that although the method of the invention is particularly useful for the generation of human antibody to a specific antigen, which antibodies could not be effectively or even feasibly obtained via immunisation of human subjects with antigen, this method is nevertheless applicable with respect to the in vitro induction of antibody production by B cells of any species including but not limited to humans, livestock animals (e.g. sheep, cows, horses, donkeys), laboratory test animals (e.g. rats, guinea pigs, rabbits, hamsters), companion animals (e.g. dogs, cats), captive wild animals (e.g. emus, kangaroos, deer, foxes) and birds (e.g. chickens, ducks, bantams, pheasants, emus, ostriches).

Preferably, said antibody producing cells are human cells.

In accordance with this preferred embodiment there is provided a method for the in vitro antigen specific activation of human B cells, said method comprising the steps of:
(i) culturing a population of isolated, non-adherent mononuclear human immune cells, which population comprises T helper cells or functional equivalent thereof, said B cells and a functionally insignificant number of lysosome-containing cells, for a time and under conditions sufficient to induce differentiation of said B cell;
(ii) culturing a population of adherent mononuclear human immune cells for a time and under conditions sufficient to facilitate antigen presenting cell differentiation;
(iii) sequentially pulsing the cell population of step (i) with:
   an effective number of cells derived from cell population of step (ii), wherein the presentation of said antigen by said antigen presenting cells is facilitated; and
      a functionally significant number of lysosome-containing cells
for a time and under conditions sufficient to facilitate antigen specific activation of said B cells;
   wherein said B cells undergo differentiation to pre-plasma cells.

Preferably, said pre-plasma cells are CD19⁺/CD38⁺.

Without limiting the present invention to any one theory or mode of action, due to the complex signalling mechanisms, both cellular and soluble, which are involved in inducing antibody production, the in vitro induction of antibody production in an antigen specific manner cannot proceed in the absence of the in vitro replication of many of these networks. Heterogeneous mononuclear immune cell populations provide a particularly advantageous naturally occurring heterogeneous cell population which can be manipulated, in accordance with the developments disclosed herein, to sufficiently replicate these mechanisms such that in vitro antigen specific B cell activation is achieved. In this regard, reference to “mononuclear immune cells” should be understood as a reference to any mononuclear cell which either directly or indirectly functions in the specific and/or non-specific immune response. Preferably, the subject mononuclear immune cells are a mononuclear cell population which has been isolated from a lymphoid organ or tissue such as blood, spleen, thymus, tonsil, lymph node or bone marrow. In a preferred embodiment, the subject mononuclear immune cells are peripheral blood mononuclear cells. In this regard, reference to “peripheral blood mononuclear immune cells” should be understood as a reference to any mononuclear cell which is either transiently or permanently located in the blood circulatory system. It should be understood that many of the mononuclear cells which can be found in the blood at any given point in time are actually recirculating cells. Accordingly, these cells are often only transiently present in the blood circulatory system and will also circulate in the lymphatic system and through the lymphoid organs (such as lymph nodes and spleen).

Methods for harvesting peripheral blood mononuclear cells would be well known to the person of skill in the art. For example, following harvesting of peripheral blood, peripheral blood mononuclear cell can be isolated by gradient separation, such as Ficoll-Hypaque centrifugation. Specifically, diluted anti-coagulated blood is layered over Ficoll-Hypaque and centrifuged. Red blood cells and polymorphonuclear leucocytes or granulocytes are more dense and centrifuge through the Ficoll-Hypaque, while mononuclear cells consisting of lymphocytes and monocytes band over it and can be recovered at the interface. Blood mononuclear cells may also be separated by elutriation, although this is not generally regarded as a preferred method of isolating such cells.

Accordingly, the present invention provides, in a preferred embodiment, a method for the in vitro antigen specific activation of human B cells, said method comprising the steps of:
(i) Culturing a population of isolated, non-adherent human peripheral blood mononuclear cells, which population comprises T helper cells or functional equivalent thereof, said B cells and a functionally insignificant number of lysosome-containing cells, for a time and under conditions sufficient to induce differentiation of said B cell;
(ii) Culturing a population of adherent human peripheral blood mononuclear cells for a time and conditions to facilitate antigen presenting cell differentiation;
(iii) sequentially pulsing the cell population of step (i) with:
   an effective number of cells derived from cell population of step (ii), wherein the presentation of said antigen by said antigen presenting cells is facilitated; and
      a functionally significant number of lysosome-containing cells
for a time and under conditions sufficient to facilitate antigen specific activation of said B cells;
   wherein said B cells undergo differentiation to pre-plasma cells.

It should be understood that although the present invention exemplifies the use of mononuclear cell populations which are freshly derived from a subject, this is not intended as a limitation with respect to the application of this invention. For example, it is also feasible that a suitable mononuclear subpopulation could have been previously isolated and stored frozen for a period of time or could be generated in vitro via combination of the requisite cellular subpopulations from distinct sources—whether they be freshly derived sources or sources such as cell lines or frozen stocks. Clearly, to the extent that cellular subpopulations comprising such a mononuclear population are not autologous, there may be issues of MHC based in vitro rejection mechanisms which would require consideration. However, the use of such mononuclear cell populations nevertheless fall within the scope of the present invention. Still further, even where a mononuclear cell population has been substantially derived from a single subject, it should nevertheless be understood that under certain circumstances it may be desirable to introduce one or more further cellular subpopulations—such as T or B cells exhibiting a particular antigen specificity, prior to commencement of the culture process of the present invention. Again, to the extent that such an additional cell subpopulation is not autologous relative to the mononuclear cell subpopulation isolated from the subject, issues of allogenicity will require consideration. However, the adaptation of the method of the present invention to overcome such problems is well within the skill of the person of ordinary skill in the art. It should also be understood that reference to “mononuclear cell population” is a reference to a whole mononuclear cell population or subpopulation thereof within the parameters defined herein. Further, it should be understood that the subject mononuclear cells may be only partially purified and may also comprise a contaminating portion of non-mononuclear cells, such as granulocytes, which nevertheless do not adversely impact on the operation of the method of the present invention. In another example, the mononuclear immune cell population of the present invention may also comprise a population of mononuclear non-immune cells which also do not adversely impact on the operation of the method of the present invention.

The mononuclear immune cell population utilised in the method of the present invention may be isolated from any suitable source and may take any initial form. For example, the cells may be isolated as a single cell suspension or as a cell aggregate such as a biopsy specimen (in which case the cells may require manipulation prior to their use in order to generate a cell suspension). The cells or aggregates may be derived from any suitable source. For example, the cells may be freshly isolated from an individual or from an existing cell line. The cells may be primary or secondary cells. A primary cell is one which has been isolated directly from a subject. A secondary cell is one which, following its isolation, has undergone some form of in vitro manipulation such as genetic manipulation. The cells may be derived directly from an individual or they may be derived from an in vitro source such as a tissue sample or organ which has been generated or synthesised in vitro. The subject cell or cell aggregate may also have been manipulated or stored subsequently to its isolation from a donor.

As detailed above, the process of the present invention may be “syngeneic”, “allogeneic” or “xenogeneic” with respect to the cellular subpopulations comprising the adherent and non-adherent mononuclear immune cell populations defined herein. Preferably, the present invention is “syngeneic”. A syngeneic process means that the individuals from which all of the mononuclear subpopulations are derived share the same MHC genotype. This will most likely be the case where whole mononuclear immune cell populations are harvested from a single individual. An “allogeneic” process is where some of the mononuclear cell sub-populations are derived from an individual who is MHC incompatible with the individual from which other components of the mononuclear cell population are derived. This may occur, for example, where the adherent and non-adherent components of the mononuclear subpopulation are isolated from distinct sources. A “xenogeneic” process is where the various mononuclear cell subpopulations are isolated from different species. For example, human derived lymphoid cells are co-cultured with non-human antigen presenting cells. Preferably, the method of the present invention is conducted as a syngeneic process although it should be understood that adapting the method such that it functions as an allogeneic or xenogeneic process may be useful in particular circumstances and is encompassed by the method of the present invention. As detailed hereinbefore, however, to the extent that either an allogeneic of xenogeneic process is utilised, it may be necessary to adapt the culture such that any immunological responses which may occur due to the mixing of foreign immunocompetent cells is minimised. It would be within the skill of the person of skill in the art to design appropriate culturing parameters.

Subsequently to isolating, and/or otherwise generating, the requisite mononuclear immune cell population, the method of the present invention defines the separate culturing of the non-adherent mononuclear immune cells from the adherent mononuclear cells. Reference to these “adherent” and “non-adherent” populations should be understood as a reference to the cellular populations which adhere to or do not adhere to, respectively, a plastic culture dish following two hours of incubation in a humidified incubator. As exemplified herein, the culture vessel is negatively charged utilising vacuum gas plasma treatment. To the extent that the cells are human, these cultures are preferably maintained in the basic culture medium of RPMI-1640, 2 mM L-glutamine and 10% FCS. It is within the skill of the person of skill in the art to adapt this method appropriately for cells derived from other species. For example, it is well known that chicken cells are required to be incubated at a higher temperature (42° C.) than mammalian cells (37° C.) due to differences in core body temperature. It should also be understood that the cells which are isolated in accordance with this aspect of the present invention may be isolated by any suitable technique which achieves the results obtainable by using the culturing method detailed above and exemplified herein.

Without limiting the present invention to any one theory or mode of action, the generation of an antigen specific B cell response, in particular to T-cell dependent antigens, requires the delivery of two signals. The first of these signals is generally delivered through the surface immunoglobulin receptor (the antigen receptor) while the second signal is delivered by T helper cells which interact with the cell surface MHCII/peptide complex on the B cell. This interaction facilitates the interaction of the T helper cell CD40 ligand with the B cell CD40 receptor and the delivery of the T helper cell derived cytokines to the B cells. Accordingly, to the extent that the present invention defines the use of a non-adherent mononuclear immune cell population, this population comprises T helper cells or functional equivalents thereof. By “T helper cell” is meant any cell expressing both a T cell receptor and the CD40 ligand. In this regard, the T cell receptor may comprise any one or more of the α, β, γ or δ chains, although cells expressing an α/β dimer are preferable. The present invention is not intended to be limited to any particular subclass of T cells, provided that the functional requirements as detailed herein are met. Preferably, the T helper cell is a T helper-2 cell (herein referred to as a “Th2” cell). Reference to a “functional equivalent” of a T helper cell should be understood as a reference to any cell which can provide any one or more of the stimulatory signals which are delivered by the T helper cell. Accordingly, there is encompassed, for example, cells which have been engineered to express the molecules required to activate B cells, but which cells do not otherwise phenotypically or morphologically correspond to classical T helper cells.

The present invention therefore preferably provides a method for the in vitro antigen specific activation of human B cells said method comprising the steps of:
(i) culturing a population of isolated, non-adherent human mononuclear immune cells, which population comprises Th2 cells or functional equivalent thereof, said B cells and a functionally insignificant number of lysosome-containing cells, for a time and under conditions sufficient to induce differentiation of said B cells;
(ii) culturing a population of adherent human mononuclear immune cells for a time and under conditions sufficient to facilitate antigen presenting cell differentiation; (iii) sequentially pulsing the cell population of step (i) with:
   an effective number of cells derived from cell population of step (ii), wherein the presentation of said antigen by said antigen presenting cells is facilitated; and
      a functionally significant number of lysosome-containing cells
for a time and under conditions sufficient to facilitate antigen specific activation of said B cells;
   wherein said B cells undergo differentiation to pre-plasma cells.

Preferably, said pre-plasma cells are CD19⁺/CD38⁺.

Preferably, said human mononuclear immune cells are peripheral blood mononuclear cells.

Reference to “lysosome-containing cells” should be understood as a reference to cells which comprise one or more intracellular lysosomes. Lysosomes are membrane bound cytoplasmic organelles which contain a range of hydrolytic enzymes that can be released into the phagosome or extracellularly. In the context of immune cells, lysosome-containing cells include, but are not limited to, phagocytic cells such as macrophages and monocytes and cytolytic cells such as CD8⁺ T cells and NK cells. In the context of the present invention, it is desirable to remove the lysosome-containing cells from the non-adherent mononuclear immune cell cultures during the early stage of the culturing process such that B cell differentiation can be initially induced. Without limiting the present invention to any one theory or mode of action, the presence of such cells at this time could result in unacceptable levels of cell death among the non-cytotoxic cell population since the lysosome-containing cells may kill cells expressing antigen. Accordingly, in order to facilitate the proliferation of the non-cytotoxic cells (such as T helper cells and B cells), the level of lysosome-containing cells must be reduced such that adverse levels of cell lysis do not occur. Reference to a “functionally insignificant number of lysosome-containing cells” should therefore be understood as a reference to the non-adherent mononuclear immune cell culture being depleted of:
(i) all lysosome-containing cells; or
(ii) at least sufficient numbers of these cells such that their adverse functional activity does not prevent or significantly retard the objective of inducing the differentiation of the antibody producing cells.

Removal of the lysosome-containing cells may be achieved by any suitable method which would be well known to those of skill in the art. The method exemplified herein is LeuLeuOMe treatment of the non-adherent population of mononuclear immune cells. This treatment does not entirely deplete the culture of lysosome-containing cells in that it has been determined, without limiting the present invention in any way, that approximately 1% of these cells escape depletion. However, it has still further been determined that this is a functionally insignificant number of lysosome-containing cells within the context of the present invention.

Reference to the induction of “differentiation” of the antibody producing cell should be understood as a reference to inducing the differentiation of immature antibody producing cells, maintaining the differentiation of antibody producing cells which have already commenced differentiation and maintaining the viability of antibody producing cells, such as memory B cells, which require ongoing antigen stimulation in order to remain viable. This objective can be achieved by any suitable means. In a preferred embodiment, it has been determined that culturing the lysosome depleted mononuclear immune cell population in the presence of suitable growth factors such as low levels of GM-CSF (in order to maintain B cell differentiation), IL-4 (to maintain Th2 proliferation) and CD40 ligand (to maintain the ongoing stimulation of memory B cells) achieves this objective. However, in light of the teachings provided herein, it would be within the skill of the person of skill in the art to modify or otherwise alter the specific culture conditions which are exemplified herein. Accordingly, it should be understood that although the present application exemplifies one set of culture conditions which have been found to be successful, this is not intended as a limitation on the scope of the culture conditions which, in light of the teachings provided herein, could be designed to similarly achieve this objective. All such culture conditions should be understood to fall within the scope of the present invention. For example, as an alternative to specifically introducing IL-4 to the culture system, one may utilise 10% v/v cell culture medium which has been harvested from a population of mononuclear cells which have been cultivated in the presence of PMA and ionomycin. Without limiting the present invention to any one theory or mode of action, cell culture medium conditioned in this way provides a suitable source of IL-4. Although this type of culture medium also contains IL-10 and IL-5, these cytokines are nevertheless known to support Th2 and B cell differentiations. Accordingly, this represents an example of an adaptation to the culture conditions expressly recited herein, which adaptation is one that could be designed by the person of skill in the art as a matter of routine procedure in light of the teachings provided herein.

The non-adherent mononuclear immune cell population is cultured under conditions sufficient to induce antigen presenting cell differentiation. As detailed hereinbefore, this culture is maintained separately to the non-adherent population of cells due to the adverse functional impact on the lymphoid cells of the culture conditions which are required to support antigen presenting cell differentiation. In this regard, reference to “antigen presenting cell” should be understood as a reference to any cell which exhibits the functional capacity to internalise foreign and/or self antigens and to present same to lymphoid cells, in particular T cells. Antigen presenting cells include, but are not limited to, dendritic cells, macrophages, B cells (in the context of presentation to T helper cells) and follicular dendritic cells (in the context of presentation t B cells). In the context of the present invention, said antigen presenting cell is preferably an antigen presenting cell which can present antigen to T helper cells and, even more particularly, is a dendritic cell. Accordingly, the present invention preferably provides a method for the in vitro antigen specific activation of human B cells said method comprising the steps of:
(i) culturing a population of isolated, non-adherent mononuclear immune cells, which population comprises Th2 cells or functional equivalent thereof, said B cells and a functionally insignificant number of lysosome-containing cells, for a time and under conditions sufficient to induce differentiation of said B cells;
(ii) culturing a population of adherent human mononuclear immune cells, for a time and under conditions sufficient to facilitate dendritic cell differentiation;
(iii) sequentially pulsing the cell population of step (i) with:
   an effective number of cells derived from cell population of step (ii), wherein the presentation of said antigen by said dendritic cells is facilitated; and
      a functionally significant number of lysosome-containing cells
for a time and under conditions sufficient to facilitate antigen specific activation of said B cells;
   wherein said B cells undergo differentiation to pre-plasma cells.

Preferably, said pre-plasma cells are CD19⁺/CD38⁺.

Preferably, said human mononuclear immune cells are peripheral blood mononuclear cells.

Reference herein to “dendritic cells” should be read as including reference to cells exhibiting dendritic cell morphology, phenotype or functional activity and to mutants or variants thereof. Reference to “dendritic cells” should be understood to include reference to cells at any differentiative state of development. The morphological features of dendritic cells may include, but are not limited to, long cytoplasmic processes, large cells with multiple fine dendrites (or other form of pseudopodia) or irregularly shaped membrane (although round cells are also observed). Phenotypic characteristics may include, but are not limited to, expression of one or more of the antigens defined by CD11c, CD123, MHC Class II, CD1, CD4, Dec205, 33D1, CD80, CD86, CD83, CMRF-44, CMRF-56, DC-SIGNE, DC-LAMP, Langerin or macrophage mannose receptors. Functional activity includes, but is not limited to, a stimulatory capacity for naive allogenic T cells, the capacity to internalise antigens and re-express peptides of said antigens in association with MHC Class II molecules. The expression of particular morphological, phenotypic and functional features will vary according to the differentiative state of the dendritic cells. For example, dendritic cell precursors are known to be effective as an antigen presenting cell. Expression of particular morphological, phenotypic and functional features may also vary between different populations of dendritic cells, such as dendritic cells arising from different cell lineages. For example, lymphoid-like dendritic cells vary from myeloid-like dendritic cells. “Variants” include, but are not limited to, cells exhibiting some but not all of the morphological or phenotypic features or functional activities of dendritic cells. “Mutants” include, but are not limited to, dendritic cells which are transgenic wherein said transgenic cells are engineered to express one or more genes such as genes encoding antigens, immune modulating agents, cytokines or receptors.

The facilitation of antigen presenting cell “differentiation” should be understood as a reference to induction of the phenotypic and/or functional maturation of an antigen presenting cell which is at any differentiative stage of development. This objective may be achieved by any suitable means, such as via growth factor stimulation. A particularly preferred means, which is exemplified herein, is to initially culture the isolated adherent mononuclear immune cell population in the presence of both IL-4 and high concentrations of GM-CSF. GM-CSF at these concentrations induces, inter alia, dendritic cell differentiation from precursor cells while IL-4 increases MHC Class II expression. This provides a pool of immature dendritic cells, which immature cells are particularly efficient with respect to the uptake and processing of antigen. The addition of TNFα to such a culture induces the differentiation of immature dendritic cells to mature dendritic cells (as evidenced by the down-regulation of CD14 expression and the up-regulation of CD83 expression), which mature dendritic cells activate T helper cells more efficiently than immature dendritic cells. Accordingly, there is also provided a means of developing a pool of mature dendritic cells suitable for T helper cell activation. Without limiting the invention in any way, the addition of INFα into the culture protocol effectively shortens the time required to generate mature dendritic cells. As detailed hereinbefore in relation to the induction of B cell differentiation, although the methods exemplified herein reflect the preferred means of inducing dendritic cell differentiation, in light of the teachings provided herein it would be within the skill of the person of skill in the art to modify or design and apply alternative culture conditions to achieve the desired objective (such as omitting TNFα use and utilising a longer culture timeframe). Accordingly, the use of any such means should be understood to fall within the scope of the method of the present invention.

Subsequent to establishing separate co-cultures of the adherent and non-adherent mononuclear immune cells, antigen specific B cell activation is achieved via a sequential series of co-culture steps. Specifically, at one or more suitable intervals an effective number of cells from the adherent mononuclear immune cell population are co-cultured with an effective number of cells from the non-adherent mononuclear immune cell population. Addition of these adherent cells to these non-adherent cells is herein referred to as “pulsing” of the non-adherent mononuclear immune cell culture. It should be understood that the establishment of this co-culture can be achieved by any suitable means which would be known to those skilled in the art including removing aliquots of cells from the primary adherent and non-adherent mononuclear cell cultures which are initially established and co-culturing these aliquots as a separate culture or introducing an aliquot of cells harvested from the primary adherent mononuclear immune cell culture directly to the primary non-adherent mononuclear cell culture. It should be understood that the present invention encompasses these and any other alternative means of establishing the subject co-cultures.

Without limiting the present invention to any one theory or mode of action, it has been determined that the adherent mononuclear immune cell population which is co-cultured with the non-adherent mononuclear immune cells, although being a heterogeneous cell population, is functionally essential due to its dendritic cell component. In this regard, it has been determined that the non-adherent mononuclear immune cell population is preferably initially pulsed with a population of adherent mononuclear immune cells comprising immature dendritic cells (these cells being particularly effective in taking up and processing antigen) followed by pulsing with a population of cells comprising more mature dendritic cells (these cells being particularly effective in activating T helper cells). In terms of these pulsing events the antigen presenting cells such as the dendritic cells, are preferably exposed to the antigen of interest prior to the sequential co-culture (pulsing) events of step (iii) in order to facilitate efficient uptake and presentation of the antigen. Means of exposing an antigen presenting cell population to the antigen of interest are well known to those of skill in the art. In the method exemplified herein, and in a preferred embodiment, this is achieved by introducing the antigen to the adherent cell culture for approximately 2 hours immediately prior to the sequential co-culturing steps. Although this method is exemplified herein, the method of the present invention should not be limited in this regard and extends to any other suitable technique of achieving antigen presentation (herein referred to as “facilitating” the presentation of said antigen by said antigen presenting cell). For example, one may elect to culture the adherent cell population with the antigen of interest for more or less than 2 hours prior to effecting the co-culturing event. Alternatively, one may introduce unprimed dendritic cells into the non-adherent cell culture simultaneously with an antigen load, thereby facilitating antigen uptake and processing by the dendritic cell population during co-culture with the lymphoid population. In yet another example, antigen may be introduced into the non-adherent cell culture prior to introduction of the adherent cell population. This facilitates the B cells which remain in the non-adherent cell population taking up the antigen in their capacity as antigen presenting cells. The T cells and antigen presenting B cells therefore interact both before and after introduction of professional dendritic cells. The subsequently introduced adherent cell population may be one which has already been exposed to antigen or it may be an unprimed population. The former scenario may be of particular use where one is seeking to achieve on going antigen uptake during the period of co-culture. This could potentially facilitate a longer period of effective antigen presentation and may be of particular value with respect to certain antigen types, the in vitro immune response to which is improved by ongoing antigen presentation.

The number and timing of these pulsing (co-culture) events may be varied according to the objective which is sought. For example, in order to obtain a population of activated but not terminally differentiated B cells, a single pulsing event may be sufficient. However, if it is desirable that antigen specific IgG secreting plasma cells are generated, multiple pulsing events (for example three as exemplified herein) will likely be required in order to push the in vitro B cell response from a primary IgM response to a secondary IgG response, to the extent that virgin as opposed to memory B cells are stimulated.

The inventors have determined that subsequently to the initial antigen presenting cell-free differentiative phase, which is performed in the absence of lysosome-containing cells, a “functionally significant” number of lysosome-containing cells are reintroduced to the non-adherent mononuclear immune cell culture. Reference to “functionally significant” number of cells should be understood as a reference to a quantity of cells which achieve the functional objective of supporting B cell activation. It has been determined that preferably up to 15% lysosome-containing cells, and preferably approximately 10% lysosome-containing cells, should be reintroduced to the non-adherent mononuclear immune cell culture. This may be conveniently achieved, for example, at the time of the first pulsing event. However, the present invention should not be limited in this regard in that the functional objective to be achieved by reintroducing these cells may nevertheless be achieved by introducing these cells simultaneously with or separately to the first pulsing event. Contrary to accepted dogma, it has now been determined that in the absence of lysosome-containing cells during the B cell activation phase, a poor immune cell response is obtained. Without limiting the present invention to any one theory or mode of action, depletion of the lysosome-containing cells (which are found in initial tissue concentrations of 30-50% depending on the tissue source which is examined) from the non-adherent mononuclear immune cell population results in depletion of the lysosome-containing cytotoxic CD8⁺ cells. It is believed that the absence of a subpopulation of the depleted CD8⁺ T cells, at the time of B cell activation, acts to adversely impact on the activation of the B cell population due to a functional contribution by these cells to this process. Accordingly, reintroduction of a small proportion of these cells has now been determined to overcome the adverse impact of their substantial absence during the B cell process. It should also be understood that in terms of the sequential pulsing event, a population of lysosome-containing cells may be reintroduced at each pulsing step or, alternatively, the lysosome-containing cells may be introduced at only some of the sequential pulsing steps to the extent that there may be functionally sufficient numbers of the lysosome-containing cells present in the culture at subsequent pulsing events thereby effectively achieving the objective as defined in accordance with the method of the present invention without necessarily reintroducing a further population. In this regard, it should also be understood that the antigen which is required to be present at each pulsing step may be achieved by incorporating exogenous antigen at each and every pulsing step or, if sufficient antigen is incorporated in one or more of the initial pulsing steps it may not be necessary to incorporate further exogenous antigen at subsequent pulsing steps in the event that sufficient antigen concentration is already present in the culture. However, it should be understood that to the extent that sufficient amounts of either antigen or lysosome-containing cells are present at the time of subsequent pulsing steps, thereby not requiring the addition of further exogenous antigen, this nevertheless satisfies the requirements for “incorporating” these molecules during each pulsing step, as detailed in step (iii) of the invention defined herein. The lysosome-containing cells may be derived from any suitable source.

Preferably, the proportions of lysosome-containing cells is approximately 10%.

Reference to “co-culture” should be understood to encompass the co-culturing of two or more populations of cells, being, in accordance with the present invention, a population of non-adherent mononuclear immune cells and a population of adherent mononuclear immune cells, each of which populations has undergone a certain degree of in vitro induced differentiation prior to the co-culturing step. It should be understood that the subject cells will have been co-cultured provided that they were at least transiently co-cultured. That is, it may be desirable to culture one or both populations of cells, in isolation, either prior or subsequently to the co-culturing step. For example, subsequently to the harvesting of pre-plasma cells, these cells may be removed from co-culture and maintained in isolation such as would occur in the event of their immortalisation.

According to a most preferred embodiment, the present invention is directed to a method for the in vitro antigen specific activation of human B cells, said method comprising the steps of:
(i) co-culturing a population of isolated, non-adherent human mononuclear immune cells, which population comprises Th2 cells or functional equivalent thereof, said B cells and a functionally insignificant number of lysosome-containing cells, together with an effective amount of one or more growth factors and anti-CD40 ligand, for a time and under conditions sufficient to induce differentiation of said B cells;
(ii) co-culturing a population of adherent human mononuclear immune cells together with an effective amount of one or more growth factors, for a time and under conditions sufficient to facilitate dendritic cell differentiation;
(iii) sequentially pulsing the cell population of step (i) with:
   an effective number of cells derived from the cell population of step (ii), wherein the presentation of said antigen by said dendritic cells is facilitated; and
   substantially 10% lysosome-containing cells;
for a time and under conditions sufficient to facilitate antigen specific activation of said B cells;
   wherein said B cells undergo differentiation to a pre-plasma cells.

Preferably said pre-plasma cells are CD19⁺/CD38⁺.

Preferably, said mononuclear immune cells are peripheral blood mononuclear cells.

More preferably, the present invention is directed to a method for the in vitro antigen specific activation of human B cells, said method comprising the steps of:
(i) co-culturing a population of isolated, non-adherent human mononuclear immune cells, which population comprises Th2 cells or functional equivalent thereof, said B cells and a functionally insignificant number of lysosome-containing cells, together with an effective amount of IL-4, GM-CSF and anti-CD40 ligand, for a time and under conditions sufficient to induce differentiation of said B cells;
(ii) co-culturing a population of adherent human mononuclear immune cells together with IL-4, GM/CSF and/or TNFα, for a time and under conditions sufficient to facilitate dendritic cell differentiation;
(iii) sequentially pulsing the cell population of step (i) with:
   an effective number of cells derived from the cell population of step (ii), wherein the presentation of said antigen by said dendritic cells is facilitated; and
   substantially 10% lysosome-containing cells;
for a time and under conditions sufficient to facilitate antigen specific activation of said B cells;
   wherein said B cells undergo differentiation to a pre-plasma cells.

The length of time for which any given co-culture is maintained prior to the instigation of a further and subsequent pulsing event will depend largely on the nature of the cell population which is utilised (for example the tissue source of the mononuclear cells or the species from which they are isolated) and the objective which is sought to be achieved. However, in light of the teachings detailed herein, determining an appropriate time course would be a matter of routine procedure for the person of skill in the art. For example, establishing whether the requisite level of differentiation has occurred could be determined by methods such as the sampling of cells from a co-culture and their phenotypic or functional analysis utilising any one of a number of routine methods which are well known to immunologists. For example, the phenotypic analysis of any given cell is most easily achieved via the performance of an indirect immunofluorescence analysis of cell surface antigen expression utilising a FACS machine. Alternatively, cells can be labelled and visualised under a microscope, such as an immunofluorescence microscope. Alternatively, various molecular techniques are available, and routinely utilised, to screen for B cell immunoglobulin gene rearrangement events. Accordingly, as a matter of routine procedure the person of skill in the art could quickly and accurately assess events relevant to adapting or refining a suitable time course such as the points in time at which there has occurred:
(i) a prescribed level of B cell differentiation;
(ii) B cell and/or T helper cell activation;
(iii) terminal B cell differentiation;
(iv) a prescribed level of differentiation of a population of dendritic cells.

Without limiting the present invention in any way, the method of the present invention is exemplified herein with respect to the in vitro induction of antigen specific pre-plasma cells from human peripheral blood or spleen derived mononuclear cells. Specifically, at day 0 the isolated mononuclear immune cell population undergoes initial culture such that the adherent and non-adherent cell populations are isolated. The non-adherent cell population is cultured together with IL-4, GM-CSF and anti-CD40 in order to induce immature B cell differentiation and to maintain memory B cell viability. The adherent cells are concurrently cultured with IL-4 and GM-CSF in order to induce differentiation of suitable precursor cells to dendritic cells. Simultaneously, a proportion of the whole mononuclear cell population is separately cultured in order to provide a source of lysosome-containing cells which are subsequently reintroduced to the non-adherent cell culture.

At day 4, an effective number of the adherent cells are introduced to the non-adherent cell cultures. These adherent cells comprise mostly immature dendritic cells which are particularly effective in processing and presenting antigen. Together with the adherent cells there is also introduced to the non-adherent cell culture the antigen in response to which the production of antibody is sought. Accordingly, dendritic cell antigen presentation to Th2 cells is effected. An unstimulated aliquot of the originally isolated adherent mononuclear immune cells is cultured with IL-4 and GM-CSF, with this culture commencing at day 4. At day 6 TNFα is added to these cultures and at day 7 these cells are introduced to the non-adherent mononuclear cell culture which had been pulsed at day 4. In addition to pulsing with the TNFα stimulated adherent cells, the non-adherent mononuclear cell culture is pulsed with further antigen. Levels of IL-4, GM-CSF and anti-CD40 are maintained. At day 8 further IL-4 and GM-CSF is added to the remainder of the TNFα stimulated adherent cell cultures and at day 11 the non-adherent mononuclear cell culture is pulsed with a further aliquot of these adherent cells together with antigen. The introduction of more mature dendritic cells facilitates Th cell activation. At day 11, the CD40 ligand is not required to be reintroduced to the culture due to sufficient upregulation of CD40 ligand expression by Th2 cells at this stage and therefore adequate CD40 stimulation of the B cells. At day 15, culture supernatants are harvested and screened for antibody production.

An “effective number” means a number of cells necessary to at least partly obtain the desired outcome. This number varies depending upon the culture conditions and other relevant factors. It is expected that the number will fall in a relatively broad range which can be determined through routine trials.

Reference to “antigen” should be understood as a reference to any proteinaceous or non-proteinaceous molecule or a derivative, homologue, analogue, chemical equivalent or mimetic thereof against which it is sought to induce a specific antibody response. In this regard, it should be understood that an antigen may comprise multiple epitopes or it may comprise a single epitope. As detailed hereinbefore, to the extent that an antigen expresses multiple epitopes it is to be expected that multiple specificities of B cells, recognising each of these epitopes, will be stimulated. To the extent that an antigen comprises a single epitope (for example a hapten), it is to be expected that a single specificity of B cell would be stimulated. Without limiting the present invention in any way, in order to stimulate a specific antibody response. it is necessary that the antigen of interest be immunogenic. In this regard, although most molecules are antigenic in that they exhibit epitopes recognised by immunointeractive molecules, some antigens (for example due to their nature and size) are not able to stimulate an immune response. Such antigens are termed “non-immunogenic”. For example, a hapten, although being antigenic, is not immunogenic and therefore must be coupled to a carrier molecule and/or multimerised in order to facilitate its recognition by the immune system and the subsequent instigation of an immune response. Assessing the immunogenicity of an antigen of interest would be well known to the person of skill in the art and means for rendering such an antigen immunogenic would be similarly a matter of routine procedure. For example, an antigen may be coupled with a carrier such as keyhole limpet cyanine (KLH) which will render a small non-immunogenic antigen immunogenic. The use of a carrier molecule to render an antigen immunogenic is likely to be of particular relevance where the antigen is a peptide.

In accordance with the present invention, the subject antigen is preferably a peptide which comprises a specific epitope for the antigen of interest and, even more preferably, a Hepatitis B or Hepatitis C virus surface antigen and, still more preferably, the 30 amino acid residue peptide coded for by the Pre-S2 region of HBV DNA (PQAMQWNSTTFHQTLQDPRVRGLYFPAGGK; SEQ ID NO: 1). Without limiting the present invention to any one theory or mode of action, since the immunodominant, disulphide bond independent epitopes recognised by human antibodies to the hepatitis B virus are more easily mimicked by peptide analogues than conformational determinants on the S gene product, the Pre-S2 region amino acid peptide has been identified as suitable for use in the method of the invention. Moreover, it has been found that the Pre-S-gene coded antigenic determinants are more immunogenic than S protein determinants located on the same HBsAg particle and immunologic non-responsiveness to the S protein can be overcome by immunisation with the Pre-S-gene coded sequences of the HBV ENV middle protein (Pre-S2+S protein).

Accordingly, in a most preferred embodiment there is provided a method for the in vitro activation of Hepatitis B specific human B cells, said method comprising the steps of:
(i) co-culturing a population of isolated, non-adherent human mononuclear immune cells, which population comprises Th2 cells or functional equivalent thereof, said B cells and a functionally insignificant number of lysosome-containing cells, together with an effective amount of IL-4, GM-CSF and anti-CD40 ligand, for a time and under conditions sufficient to induce differentiation of said B cells;
(ii) co-culturing a population of adherent human mononuclear immune cells together with IL4, GM-CSF and/or TNFα, for a time and under conditions sufficient to facilitate dendritic cell differentiation;
(iii) sequentially pulsing the cell population of step (i) with:
   an effective number of cells derived from the cell population of step (ii), wherein the presentation of Pre-S2 HBV by said dendritic cells is facilitated; and
   substantially 10% lysosome-containing cells;
      for a time and under conditions sufficient to facilitate antigen specific activitation of said B cells;
      wherein said B cells undergo differentiation to pre-plasma cells.
      wherein said B cells undergo differentiation to pre-plasma cells.

Reference to “Pre-S2 HBV” should be understood to encompass reference to derivatives, homologues, analogues, chemical equivalents and mimetics of said antigen.

In another preferred embodiment, the subject antigen is the tetanus toxoid precursor₈₂₉₋₈₄₃ (QYIKANSKFIGITEL; SEQ ID NO:2), Melan A₂₇₋₃₅(AAGIGILTV; SEQ ID NO:3), HIV-1 gp120₃₀₄₋₃₁₈ (RKSIRIQRGPGRAFV; SEQ ID NO: 4)HIV-1 gp120₂₉₄₋₄₇₃, HER2₃₆₉₋₃₇₇ (KIFGSLAFL; SEQ ID NO:5).

Derivatives of the nucleic acid and protein molecules defined herein include fragments, parts, portions, mutants, variants and mimetics from natural, synthetic or recombinant sources including fusion proteins. Parts or fragments include, for example, active regions. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, adenomaeptides or proteins.

Homologues should be understood as a reference to nucleic acid or protein molecules isolated from or otherwise corresponding to molecules found in species other than the human.

Chemical and functional equivalents of the subject nucleic acid or protein molecules should be understood as molecules exhibiting any one or more of the functional activities of these molecules and may be derived from any source such as being chemically synthesized or identified via screening processes such as natural product screening.

The derivatives include fragments having particular epitopes or parts of the entire protein fused to peptides, polypeptides or other proteinaceous or non-proteinaceous molecules.

Analogues contemplated herein include, but are not limited to, modification to side chains, incorporating of unnatural amino acids and/or their derivatives during peptide, adenomaeptide or protein synthesis and the use of crosslinkers and other methods which impose conformational constraints on the proteinaceous molecules or their analogues.

Derivatives of nucleic acid sequences may similarly be derived from single or multiple nucleotide substitutions, deletions and/or additions including fusion with other nucleic acid molecules. The derivatives of the nucleic acid molecules of the present invention include oligonucleotides, PCR primers, antisense molecules, molecules suitable for use in cosuppression and fusion of nucleic acid molecules. Derivatives of nucleic acid sequences also include degenerate variants.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 2.

TABLE 2

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl--aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanin | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2- | Nmbc | | |
| diphenyl-ethylamino)cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

The methods and compositions of the present invention are useful for generating both activated B cells and antibody, in an antigen specific manner, for use in a range of prophylactic, therapeutic and diagnostic procedures. In particular, the development of an in vitro system for producing antibodies in an antigen specific manner now facilitates the routine production of human antibodies. To date, the use of non-human antibodies for human therapeutic and prophylactic applications has often resulted in the generation of the HAMA response. Efforts have been made to overcome these difficulties by humanising what would otherwise be rodent-derived antibodies. However, this procedure is complex and expensive.

In considering the potential applications of the activated B cells and antibodies which are generated in accordance with the method of the present invention, it should be understood that at the conclusion of the cell culture method disclosed herein, one is in possession of a heterogeneous population of cells, of which only a small proportion represent the differentiated B cells of interest, and a culture supernatant which effectively comprises a population of polyclonal antibodies. As detailed hereinbefore, although it is possible that in response to a single epitope, such as a hapten, stimulation and activation of a single specificity of B cell may be obtained, to the extent that an antigen expresses more than one epitope it is feasible that more than one specificity of antibody will have been stimulated. Accordingly, although there are some circumstances in which a polyclonal supernatant can be utilised, in general it is desirable to establish a monoclonal culture such that a singular specificity of antibody generated by a single B cell is produced.

Accordingly, the present invention should be understood to extend to the generation of clonal cultures which facilitate the expression of a monoclonal antibody. This is usually achieved by isolating a single pre-plasma cell which produces an antibody directed to the antigen specificity of interest and expanding this cell in vitro in order to form a cell line. However, due to the inherent limited lifespan of non-neoplastic cells, it will be desirable to immortalise the pre-plasma cell of interest in order to provide an ongoing source of the subject antibody. This is of particular relevance where commercial quantities of antibodies are required to be produced, therefore requiring the expansion of the subject pre-plasma cell number on a large scale in order to facilitate ongoing production of adequate quantities of the antibody of interest. The immortalisation of B cells is a technique which is well known in the art and could be achieved as a matter of routine procedure by the person of skill in the art. In general, the pre-plasma cell of interest is fused with an immortal cell line (such as a neoplastic cell line). Examples of methods for immortalising cells include, but are not limited to, the methods detailed in Olsson et al. (1980) Abrams et al (1983) and Abraham et al. (2001).

In terms of the means by which such immortalisation is achieved, the present invention extends to the application of a large range of techniques which are available to the person of skill in the art. For example, isolating a pre-plasma cell expressing antibody of a particular specificity may be achieved via fluorescence activated cell sorting which is performed utilising the antigen of interest, which antigen has been coupled to a fluorescent marker such as FITC. By isolating such a cell a single cell fusion could then be performed or, if the cell is isolated under sterile conditions and cultured in order to achieve expansion in number, a bulk fusion may be performed. There are particular advantages associated with a bulk fusion since not every fusion event is successful. Accordingly, where a bulk fusion has been performed on a clonal population of cells the subsequently resulting fused cell population can be screened in order to identify those cells which are the strongest producers of antibodies.

Another alternative is to conduct a bulk fusion of the B cell/pre-plasma cell population which is derived from the culture method of the present invention. Subsequently to this fusion event it would be necessary to screen the fused cells in order to identify which cell, if any, is producing the antibody of interest. The choice of method will depend largely on the circumstances in any given situation and can be determined as a matter of routine procedure by the person of skill in the art.

In addition to the notion of fusing a pre-plasma cell with an immortalised cell line (such as myeloma cell lines of B lymphablastoid cell lines), being the traditionally accepted means of immortalising pre-plasma cells, there are also available other techniques for immortalising cells. These include for example the viral (e.g. EBV) transformation of a cell or the use of an oncogene expression construct. Numerous means of effecting viral transformation have been developed including co-culturing of cells with viral particles or their introduction via cellular electroporation. Alternatively, the genetic manipulation of cells, utilising recombinant techniques, may provide a means of introducing a mutation which would result in immortalisation of a cell of interest.

In another example, the on-going availability of an antibody specificity of interest could be achieved by isolating the nuclear material from the pre-plasma cell which produces the subject antibody and creating a phage display library which thereby enables the generation of a clone expressing the recombinant antibody or derivative or fragment thereof.

In terms of the application of the antibodies which are generated in accordance with the method of the present invention, there is an extensive range of potential therapeutic and prophylactic applications including, but not limited to, therapeutic treatment for acute or chronic pathogenic infections, the use of antibodies as “magic bullets” to administer any proteinaceous or non-proteinaceous molecule in a site directed mechanism and to rapidly and efficiently neutralise toxins such as those generated by insects and arachnids. The method of the present invention can also be used to provide specific immunized autologous cells such as CD4⁺ T cells, CD8⁺ T cells or B cells for treating disease conditions such as HIV, Hepatitis C, allergy or cancer.

Another aspect of the present invention should therefore be understood to extend to a method of therapeutically and/or prophylactically treating a subject, said method comprising administering to said subject an effective amount of antibody or derivative, homologue, analogue, chemical equivalent or mimetic of said antibody wherein said antibody is produced by the method of the present invention.

Reference herein to “therapeutic” and “prophylactic” treatment is to be considered in its broadest context. The term “therapeutic” does not necessarily imply that a subject is treated until total recovery. Similarly, “prophylactic” does not necessarily mean that the subject will not eventually contract a disease condition. Accordingly, therapeutic and prophylactic treatment includes amelioration of the symptoms of a particular condition or preventing or otherwise reducing the risk of developing a particular condition. The term “prophylactic” may be considered as reducing the severity or the onset of a particular condition. “Therapeutic” may also reduce the severity of an existing condition.

The subject of the treatment or prophylaxis is generally a mammal such as but not limited to human, primate, livestock animal (e.g. sheep, cow, horse, donkey, pig), companion animal (e.g. god, cat), laboratory test animal (e.g. mouse, rabbit, rat, guinea pig, hamster), captive wild animal (e.g. fox, deer). Preferably the mammal is a human or primate. Most preferably the mammal is a human.

Administration of the antibody of the present invention in the form of a pharmaceutical composition, may be performed by any convenient means. The antibody of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 microgram to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The antibody may be administered in a convenient manner such as by the oral, intravenous (where water soluble), intraperitoneal, intramuscular, subcutaneous, intradermal or suppository routes or implanting (e.g. using slow release molecules).

In accordance with these methods, the antibody defined in accordance with the present invention may be coadministered with one or more other compounds or molecules. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

In another aspect, the present invention contemplates a pharmaceutical composition comprising an antibody as hereinbefore defined and one or more pharmaceutically acceptable carriers and/or diluents. Said antibodies are referred to as the active ingredients.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding th subject antibody. The vector may, for example, be a viral vector.

Still another aspect of the present invention is directed to the antibody or derivative, homologue, analogue, equivalent or mimetic and activated antibody producing cells produced in accordance with the methods defined hereinbefore It should be further understood that the subject antibody producing cell may be one which has been immortalised or otherwise manipulated.

In another aspect, the molecules of the present invention are also useful for screening for the antigens against which they are directed, such as in the context of the diagnosis of disorders characterised by expression of the antigen. The screening methodology may be directed to qualitative and/or quantitative analysis.

Screening for the subject antigens in a sample, such as a biological sample, can be performed by any one of a number of suitable methods which are well known to those skilled in the art. For example, the presence of an antigen may be determined in a number of ways such as by Western blotting, ELISA or flow cytometry procedures. These, of course, include both single-site and two-site or “sandwich” assays of the non-competitive types, as well as in the traditional competitive binding assays. These assays also include direct binding of a labelled antibody to a target.

Sandwich assays are among the most useful and commonly used assays and are favoured for use in the present invention. A number of variations of the sandwich assay technique exist, and all are intended to be encompassed by the present invention. Briefly, in a typical forward assay, the unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent. In accordance with the present invention the sample may be tested includes cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

In the typical forward sandwich assay, the antibody of the present invention, or antigenic parts thereof, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes) and under suitable conditions (e.g. 25° C.) to allow binding of any subunit present in the antibody. Following the incubation period, the antibody subunit solid phase is washed and dried and incubated with a second antibody specific for a portion of the hapten. The second antibody is linked to a reporter molecule which is used to indicate the binding of the second antibody to the hapten.

An alternative method involves immobilizing the target molecules in the biological sample and then exposing the immobilized target to the antibody of the invention which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells on latex beads, and the like.

Alternately, fluorescent compounds, such as fluorecein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength the fluorescence observed indicates the presence of the hapten of interest. Immunofluorescene and EIA techniques are both very well established in the art and are particularly preferred for the present method. However, other reporter molecules, such as radioisotope, chemiluminescent or bioluminescent molecules, may also be employed.

The present invention is further defined by the following non-limiting Examples.

### EXAMPLE 1

### Materials

Pre S2 peptide (SEQ ID NO:1). Synthesised by Auspep

1 mg/ml in 10 mM Hepes pH 5.4, 29 amino acids,

| | | Catalogue |
|---|---|---|
| Reagent | Manufacturer/Supplier | number |
| Ficoll-Paque Plus | Pharmacia-Biotech | 17-0840-02 |
| RPMI1640 | Gibco | 13200-076 |
| Penicillin-streptomycin | CSL | 05081901 |
| PreS2 | Auspep (synthesis as | Batch K31114 |
| (1 mg/ml in 10 mM Hepes | request sequences) | |
| pH 5.4, 29 amino acids) | | |
| rhGM-CSF | R&D Systems | 215-GM |
| rhIL-4 | Sigma | I-4269 |
| rhIL-10 | Bio-scientific Pty Ltd | 217-IL |
| Anti-human CD40 | Becton Dickinson Pty Ltd | 550391 |
| KLH Immunogen | Pierce | 77608 |
| Conjugation Kit | | |
| Tween-20 | ICN | 103168 |
| Streptavidin | Bio-Rad | 170-6408 |
| Biotinylated Alkaline | Bio-Rad | 170-6403 |
| Phosphatase | | |
| Human IgA | Sigma | I2636 |
| Human IgG | ICN | 64145 |
| Human IgM | ICN | 65345 |
| Leu-leu Methyl Ester, | ICN | 153142 |
| Hydrobromide (MW 339.3) | | |
| 6 well plate | Becton Dickinson (Falcon) | 353046 |

ECM contains:
RPMI 1640
10% FCS
5% Sodium Pyruvate
5% Non-essential Amino Acids
2 mM L-Glutamine
10 mM HEPES
   Method
   Day 0
   Isolation of Lymphocytes from Tissue
   Spleen Tissue Treatment:
Cut the spleen tissue into small pieces and mash it with a syringe plunger and gently push the cells through a sterilized sieve in PBS
Filter the cell suspension with sterilized gauze and then 100 μM pore size nylon mesh to remove the lumpy tissue before diluting the cells at ˜1×10⁸/ml in PBS (no Ca⁺⁺ and no Mg⁺⁺)
Centrifuge with 10 ml underlaid Ficoll histopaque at 480 g for 30′ without brake on.
Mononuclear cells are obtained by removing the creamy layer after the centrifuge and wash in PBS 3 times
   Buffy Coat Treatment:
Dilute every 10 mls of Buffy Coat obtained as defined above up to 35 mls with PBS (no Ca⁺⁺ and Mg⁺⁺) in a 50 ml of Falcon centrifuge tube
Underlay 10 mls of Ficoll paque
Centrifuge the fractions at 480 g for 30′ without brake on
Mononuclear cells are obtained by removing the creamy layer after the centrifuge. Wash in PBS 3 times
   Take 2×10⁷ cells to proceed to PMA (10 ng/ml)+ionomycin (1 μg/ml) stimulation.
      Suspend 2×10⁷ mononuclear cells in 6 ml RPMI 1640+2 mM L-glutamine+5 ml Sodium Pyruvate+5 ml non-essential amino acids+4 μl 2me+Pen/Strep+10% heat inactivated FCS containing PMA and ionomycin. Cells are incubated in a 90 mm petri dish for 4 days.
   Keep 1.5×10⁸ cells in RPMI 1640+2 mM L-glutamine+10% heat inactivated FCS
      Isolation the Monocytes
Resuspend cells in RPMI 1640+2 mM L-glutamine+10% heat inactivated FCS at 1×10⁷/ml
Dispense 2 mls per well in 6-well plate
Incubate the plate at 37° C. for 2 hours
After 2 hours, collect the nonadherent cells from each well and wash each well with warm (37° C.) PBS (no Ca⁺⁺ and no Mg⁺⁺)
Perform LeuLeuOMe treatment, as detailed below
   Adherent Cell Treatment
Add 2 ml fresh medium (ECM+2me+Pen/Strep) to each well of 6-well plate
Label the cultures as DC1, DC2, DC3, DC4, DC5 and DC6 on one 6-well plate
Add 60 μl of GM-CSF (final at 30 ng/ml) and 60 μl of IL4 (final at 30 ng/ml) to each well with the adherent cells
   LeuLeuOMe Treatment of the Monocyte Population
Make up cell suspension at 5×10⁶/ml in FCS free medium
Add 2.5 mM (0.85 μl/ml) to culture and gently mix
Incubate at room temperature for 15 min
Wash with FCS free medium twice and resuspend in 10 mls 10% FCS+RPMI culture medium
Incubate at 37° C. for 45 min and add 20 μl (2 μl/ml) DNAseI to the culture for further 15 min incubation
Wash the cells with RPMI or PBS three times
Ready for culture
Plate the LeuLeuOMe treated cells in 96-well round bottom plate at 5×10⁵/100 μl/well containing GM-CSF 5 ng/ml, IL-4 5 ng/ml, antiCD40 0.5 μg/ml and 5 μg/ml PreS2 (the antigen)
   Day 4
Scrape cells from DC1 and DC2 cultures into one tube, count the cells, divide into 2 parts in two different tubes.
Spin both tubes at 1500 rpm for 5 min
Re-suspend cells in ˜200 μl of the supernatants respectively
Add PreS2 (at 5 μg/ml) and KLH-PreS2 (at 10 μg/ml), to tube 2 but leave tube 1 with no antigen added
Incubate the two tubes in TC incubator with lid loose for 2 hours.

Remove all cells that are stimulated by PMA+ionomycin in a tube
Spin down cells at 1500 RPM for 5 min and keep the supernatant
   Add antiCD40 at 0.5 μg/ml to the supernatant of the cell cultures that were previously stimulated with PMA+ionomycin
   Cells are re-suspended in above supernatant separately
   Remove 10 μl from each well of the 96-well plates that had LeuleuOMe treated mix cells
   Dispense 10 μl per well of above tube 1 (no Ag added) to Culture 1 and tube 2 (with Ag) to culture 2 the 96-well plates

To the rest of the adherent cells
Add GM-CSF and IL-4 at 30 ng/ml each to DC3, DC4, DC5, DC6
   Day 6
Add TNFα at 100 units/ml to cultures DC3, DC4, DC5 and DC6
   Day 7
Repeat day 4 but using DC3, DC4 instead of DC1, DC2 and use 50 μl per well in RPMI 1640+5 ml Sodium Pyruvate+5 ml non essential amino acids+2me+1% Pen/strep+10% FCS+20 μg/ml human transferrin containing 2 ng/ml IL4 and 2 ng/ml GMCSF
   Day 8
Add IL-4 and GM-CSF (30 ng/ml each) to DC5, DC6
   Day 11
Repeat day 7 immunisation but use DC5, DC6
To each well dispense 30 μl of the DCs in RPMI 1640+5 ml sodium pyruvate+5 ml non-essential amino acids+2me+1% Pen/strep+10% FCS+20 μg/ml Human Transferrin containing 2 ng/ml IL4 and 2 ng/ml GMCSF
   Day 15

Perform ELISPOT and store supernatant for ELISA

### EXAMPLE 2

### Immunisation of Spleen Lymphocytes

### sp139 Mi

This experiment was an in vitro immunisation experiment carried out on splenocytes, from which a proportion of monocytes were isolated on day 0. On day 4, 7 and 10 autologous DCs that were derived from the monocytes were added back to the other mononuclear cells that had been treated with LeuLeuOMe. The culture medium was generated from autologous NK, CD8 and other T cells under PMA stimulation, the LeuLeuOMe treated cells were cultured in the supernatant of the above cells.

Day 0

Thawed four vials of frozen cells (1×10⁸ cells/vial), assume 55% viable cell per vial. ˜2.0×10⁸ may be obtained
Obtained 5.56×10⁷ cells
   PMA Stimulation
Suspended 2×10⁷ mononuclear cells in 4 ml culture medium+4 μl 2me+40 μl Pen/Strep and PMA 50 ng/ml
Dispensed 2 ml per well to a 6-well plate
Incubated the plate in a TC incubator at 37° C. overnight
   Monocyte Isolation
Resuspended the rest of the cells in 12 ml of culture medium
Dispensed 2 ml per well in a 6-well plate at 3×10⁶/ml and incubated at 37° C.
After 2 hours, non-adherent cells were removed
Added 2 ml of fresh medium (ECM+2me+Pen/Strep) containing 30 ng/ml GM-CSF and IL4 to each well
Incubated the plate in the TC incubator until day 4.
   Day 1

To PMA stimulation culture
Removed PMA by washing with culture medium
Resuspended the cells into 4 mls of fresh ECM+2me+Pen/Strep and dispensed in 6-well plate at 2 ml per well
Continued to incubate the cells in a TC incubator until day 4 when they were ready to use
   Day 4

Thawed 4 vials.
Performed leuleuOMe treatment to remove lysosome-rich cells as described in Example 1
4.06×10⁷ cells were obtained

Kept 2×10⁶ cells to be stained (see table below)

| Markers analysed | % |
|---|---|
| mlgG1 FITZ/PE/DB | 0.13/1.12/0.37 |
| CD4PE | 23.5 |
| CD4PE/CD69FITC | 4.72 |
| CD8PE | 17.22 |
| CD8PE/CD69FITC | 3.18 |
| CD14 | 3.18 |
| CD19PE | 42.42 |
| CD19PE/CD69FITC | 0 |
| CD38 Cychrome | ? |
| CD56FITC | 2.78 |
| CD83FITC | 0.14 |
| CD1aFITC | 0 |
| Auto PEFITC | |

Plated the rest of the cells in a 96-well plate at 5×10⁵/100 μl/well with 8 mls of supernatant of PMA stimulated culture plus GM-CSF 5 ng/ml, IL-4 5 ng/ml, IL-10 20 ng/ml, Pen/Strep 1:100 and anti-CD40 0.5 μg/ml.
Scraped cells from 4 wells of monocyte culture, thereafter cells were named as DC1 and DC2
Split cells into 2 groups in different tubes and spun down at 1500 rpm for 5 min.
Resuspended the cells in ˜200 μl of existing supernatants respectively
Added 1 μl 1 mg/ml PreS2 (5 μg/ml) to DC1 and 3 μl of KLH-PreS2 (10g/ml) to DC2
Incubated the 2 tubes in a TC incubator with lid loose for 2 hours.
Counted the cells after the 2 hour incubation
Washed all cells twice in cold ECM
Culture 1: 1×10⁶ DC1 were resuspended in 1 ml of PMA stimulated culture supernatant and mixed with the LeuLeuOMe treated mononuclear cells at 40 μl/well Culture 2: DC2 was mixed with the LeuLeuOMe treated mononuclear cells
1.0×10⁶ DC1 were resuspended in 1 ml of the above supernatant 3.2×10⁶ DC2 were resuspended in 2.2 ml of the above supernatant
Dispensed 40 μl of above DC1 and DC2 to the 96-well plate respectively as designed below

[image]

To the rest of the adherent cells:
GM-CSF and IL-4 at 30 ng/ml each were added
   Day 6
Added 1 μl/ml of 10 μg/ml TNFα to the adherent cell culture.
   Day 7
Thawed 2 vials of SP139 splenocytes
5.2×10⁷ cells were obtained
Performed LeuLeuOMe treatment as described above
Mixed with DC3 and dispensed in a 96-well plate (20 wells formed) as a negative control that had no antigen added.
DC4 was pulsed with PreS2 and PreS2-KLH for 2 hours and then dispensed at 50 ul per well to the plate (see below) [image]
Another 20 wells were set up with LeuLeuOMe and 2 vials of newly thawed cells from SP139. This will be represented as a negative control that contained no antigen.
   Day 8
Added IL-4 and GM-CSF (30 ng/ml each) to last 4 well adherent cell lots
   Day 11

Performed FACS staining on cultures of DCs+MLCs.

| | DC1(PreS2)-D4 | DC2(KLH-PreS2)-D4 |
|---|---|---|
| | MLC | MLC |
| Markers | DC3(KLH-PreS2)D7 (%) | DC4(KLH-PreS2)D7 (%) |
| MlgG PE/FITC | 1.23/1.11/1.16 | 3.31/3.09/5.51 |
| CD4PE | 21.19 | 16.34 |
| CD4PE/CD69FITC | 4.18 | 1.27 |
| CD8PE | 9.48 | 9.02 |
| CD8PE/CD69FITC | 0.98 | 0.87 |
| CD19PE | 21.47 | 3.92 |
| CD19PE/CD69FITC | 1.47 | 0.08 |
| CD1a | 0.03 | 0 |
| CD83FITC | 3.62 | 1.85 |
| Annexin V | 0 | 0 |
| 7AAD | 44.09 | 47.99 |

Collected cells from 6 wells of each culture in the 96-well plates.
Washed for ELISPOT
Repeated day 4 immunisation but used rest of adherent cells—DC5, DC6
Dispensed 50 μl/well as shown below [image]
   ELISPOT Results:
No anti-PreS2 antibody secreting cells were detected.
   Day 14
   Performed ELISPOT

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Abraham Karpas Alla Dremucheva Barbara H Czepulkowski, 2001. Proc. Natl. Acad. Sci 98:(4) 1799-1804
Paul G Abrams, James A Knost, Gregory Clarke, Shannon Wilburn, Robert K Oldham, and Kenneth A Foon, 1983. the Journal of Immunology 131: (3)1201-1204
Akbar A N, Salmon M, Savill J, Janossy G, 1993. Immunol. Today 14, 526
A De Boer, Y C M Kruize and M Yazdanbakhsh, 1998. Clin Exp Immunol 114: 252-257.
Sarah Drayton and George Peters, 2002. Current Opinion in Genetics & Development 12: 98-104
Köhler, G. and Milstein, C., 1975. Nature 256: 495-497
Lane P, Traunecker A, Hubele S et al. 1992. Eur J Immunol 22:2573-2578
Noelle R J, Ledbetter J A, Aruffo A, 1992. Immunol Today 13:431-433
Olsson L and H S Kaplan, 1980. Proc. Natl. Acad. Sci. USA 77:5429
Rousset F, Garcia E, Banchereau J, 1991. J Exp Med 173:705-710
Enno Schmidt, Ulrich Leinfelder et al., 2001. J Immunol. Methods 255:93-102
Thurner B, Roder C, Dieckman D et al, 1999. J. Immunol. Methods 223, 1
Jeffrey Winkelhake (Emeryville, Calif.), 1988. Myelomas for producing human/human hybridomas.

1. A method for the in vitro antigen specific activation of antibody producing cells, said method comprising the steps of:
(i) culturing a population of isolated, non-adherent mononuclear immune cells, which population comprises T helper cells or functional equivalent thereof, said antibody producing cells and a functionally insignificant number of lysosome-containing cells, for a time and under conditions sufficient to induce differentiation of said antibody producing cell;
(ii) culturing a population of adherent mononuclear immune cells for a time and under conditions sufficient to facilitate antigen presenting cell differentiation;
(iii) sequentially pulsing the cell population of step (i) with:
an effective number of cells derived from cell population of step (ii), wherein the presentation of said antigen by said antigen presenting cells is facilitated; and
a functionally significant number of lysosome-containing cells
for a time and under conditions sufficient to facilitate antigen specific activation of said antibody producing cells.

2. The method according to claim 1 wherein said antibody producing cell is a B cell.

3. The method according to claim 2 wherein said B cells are human B cells.

4. The method according to claim 1 wherein said antigen presenting cell is a dendritic cell.

5. The method according to claim 2 wherein said activated B cells undergo differentiation to pre-plasma cells.

6. The method according to claim 5 wherein said pre-plasma cell is a CD19⁺/CD38⁺ cell.

7. The method according to claim 1 wherein said mononuclear immune cells are isolated from a lymphoid organ or tissue.

8. The method according to claim 7 wherein said lymphoid organ or tissue is blood, spleen, thymus, tonsil, lymph node or bone marrow.

9. The method according to claim 8 wherein said lymphoid tissue is blood.

10. The method according to claim 9 wherein said blood is peripheral blood.

11. The method according to claim 2 wherein said T helper cell is a Th2 cell.

12. The method according to claim 1 wherein said functionally significant number of lysosome-comprising cells is up to 15% lysosome containing cells.

13. The method according to claim 12 wherein said functionally significant number of lysosome-containing cells is up to 10% lysosome containing cells.

14. The method according to claim 2 wherein:
(a) the mononuclear immune cells, B cells and functionally insignificant number of lysosome containing cells of step (i) are cultured together with an effective amount of one or more growth factors and anti-CD40 ligand; and
(b) said adherent mononuclear immune cells of step (ii) are cultured together with one or more growth factors.

15. The method according to claim 14 wherein the growth factors of step (i) are IL-4 and GM-CSF.

16. The method according to claim 15 wherein the growth factors of step (ii) are IL-4, GM-CSF and/or TNF-α.

17. The method according to claim 2 wherein said antigen is a peptide comprising at least one epitope.

18. The method according to claim 17 wherein said epitope is an epitope of a Hepatitis B or Hepatitis C virus surface molecule.

19. The method according to claim 18 wherein said Hepatitis B epitope is the 30 amino acid residue peptide coded for by the Pre-S2 region of HBV DNA.

20. The method according to claim 19 wherein said epitope substantially corresponds to the sequence:

| | |
|---|---|
| PQAMQWNSTTFHQTLQDPRVRGLYFPAGGK | (SEQ ID NO:1) |

21. The method according to claim 17 wherein said peptide comprises an epitope of the tetanus toxoid precursor.

22. The method according to claim 21 wherein said epitope comprises the tetanus toxoid region defined by residues 829-843.

23. The method according to claim 22 wherein said epitope substantially corresponds to the sequence:

| | |
|---|---|
| QYIKANSKFIGITEL | (SEQ ID NO:2) |

24. The method according to claim 17 wherein said peptide comprises an epitope of Melan A.

25. The method according to claim 24 wherein said epitope comprises the Melan A region defined by residues 27-35.

26. The method according to claim 25 wherein said epitope substantially corresponds to the sequence:

| | |
|---|---|
| AAGIGILTV | (SEQ ID NO:3) |

27. The method according to claim 17 wherein said peptide comprises an epitope of HIV1 gp120.

28. The method according to claim 27 wherein said epitope comprises the HIV1 gp120 region defined by residues 304-318

29. The method according to claim 28 wherein said epitope substantially corresponds to the sequence:

| | |
|---|---|
| RKSIRIQRGPGRAFV | (SEQ ID NO:4) |

30. The method according to claim 27 wherein said epitope comprises the HIV1 gp120 region defined by residues 294-473.

31. The method according to claim 17 wherein said peptide comprises an epitope of HER2.

32. The method according to claim 31 wherein said epitope comprises the HER2 region defined by residues 369-379.

33. The method according to claim 32 wherein said epitope substantially corresponds to the sequence:

| | |
|---|---|
| KIFGSLAFL | (SEQ ID NO:5) |

34. Cell cultures produced in accordance with the method of claim 1.

35. The antibody producing cells generated in accordance with the method of claim 1.

36. The cells according to claim 35 wherein said cells are clonal.

37. The cells according to claim 35 wherein said cells are immortal.

38. The antibody produced by the cells of claim 35 or derivative, homologue, analogue or mimetic of said antibody.

39. A pharmaceutical composition comprising the antibody of claim 38 together with one or more pharmaceutically acceptable carriers and/or diluents.

40. A method of therapeutically and/or prophylactically treating a subject, said method comprising administering to said subject an effective amount of antibody according to claim 38 or derivative, homologue, analogue, chemical equivalent or mimetic of said antibody.

41. A method of detecting an antigen, said method comprising contacting a putative antigen with an antibody directed to an epitope of said antigen, which antibody has been generated in accordance with the method of claim 1 and screening for the formation of an antigen-antibody complex.


SEQUENCE LISTING (5 sequences)

SEQ ID NO: 1 - protein, 30 aa, Hepatitis B
PQAMQWNSTTFHQTLQDPRVRGLYFPAGGK

SEQ ID NO: 2 - protein, 15 aa, Tetanus toxoid
QYIKANSKFIGITEL

SEQ ID NO: 3 - protein, 9 aa, Melan A
AAGIGILTV

SEQ ID NO: 4 - protein, 15 aa, HIV I
RKSIRIQRGPGRAFV

SEQ ID NO: 5 - protein, 9 aa, HER2
KIFGSLAFL